(19) United States
(12) Patent Application Publication
(10) Pub. No.: US 2017/0323051 A1
(43) Pub. Date: Nov. 9, 2017

(54) DEVICES, SYSTEMS, AND METHODS FOR HIGH-RESOLUTION MELT ANALYSIS

(71) Applicant: Canon U.S. Life Sciences, Inc., Rockville, MD (US)

(72) Inventors: Sophie Isabelle Marie Paquerault, Rockville, MD (US); Bradley Scott Denney, Irvine, CA (US)

(21) Appl. No.: 15/239,495

(22) Filed: Aug. 17, 2016

Related U.S. Application Data
(60) Provisional application No. 62/206,241, filed on Aug. 17, 2015.
(60) Provisional application No. 62/353,602, filed on Jun. 23, 2016.

(51) Int. Cl. G06F 19/20; G06F 19/24; C12Q 1/68; B01L 7/00

(52) U.S. Cl. CPC G06F 19/20; C12Q 1/6816; G06F 19/24; B01L 7/52; C12Q 2539/10

(57) ABSTRACT

Devices, systems, and methods for automatic genotyping obtain high-resolution melt data from a test sample defining a melting curve for a target nucleic acid in the test sample; obtain high-resolution melt data from a control sample defining a melting curve for a wild type of the target nucleic acid in the control sample; calculate melting curve derivatives of the melting curves for the test sample and the control sample, respectively, wherein each melting curve derivative represents a negative derivative of a fluorescence emitted from a nucleic acid sample as a function of temperature affecting nucleic acid denaturation; calculate parameters defining differences between features of the test sample and the control sample melting curve derivatives; and assign a genotype to the test sample based on a comparison of the calculated parameters to predetermined thresholds and boundaries defining genotypes.

### CROSS-REFERENCE TO RELATED APPLICATIONS

This application claims the benefit of U.S. Provisional Application No. 62/206,241, which was filed on Aug. 17, 2015, and the benefit of U.S. Provisional Application No. 62/353,602, which was filed on Jun. 23, 2016, both of which are hereby incorporated by reference.

### BACKGROUND

### Technical Field

This application generally relates to high-resolution melt (HRM) analysis of deoxyribonucleic acid (DNA) samples.

### Background

Some techniques that are used to detect small quantities of nucleic acids replicate some or all of a nucleic acid sequence many times, and the amplified products can be analyzed more easily. Polymerase chain reaction (PCR) is an example of these amplification techniques. PCR can be used to amplify sections of deoxyribonucleic acid (DNA), and PCR can quickly produce millions of copies of DNA starting from a single template DNA molecule.

Once PCR has successfully generated a sufficient number of copies of the DNA section(s) of interest, the DNA section(s) can be characterized. For example, the genotype of the DNA section(s) can be determined (i.e., one or more altered nucleic acids or mutations on the DNA section(s) can be detected). One method of characterizing the DNA examines the DNA's dissociation behavior as the DNA transitions from double-stranded DNA (dsDNA) to single-stranded DNA (ssDNA) while the sample is heated with successively increased temperatures. The process of causing DNA to transition from dsDNA to ssDNA and monitoring such a transition on a fine temperature scale (e.g., every 0.01° C. on a defined temperature range) may be referred to as a high-resolution temperature (thermal) melt (HRTm) process or a high-resolution melt (HRM) process.

In HRM, two strands of nucleic acid are denatured in the presence of a dye that indicates whether the two strands of nucleic acid are bound (e.g., dsDNA) or not (e.g., ssDNA). As the temperature of the sample is raised, a reduction in fluorescence from the dye indicates that the two strands of nucleic acid have partially or completely dissociated (i.e., unzipped to single strands). Thus, by measuring the dye fluorescence as a function of temperature, features associated with one or more nucleic acids in the two strands can be obtained.

### SUMMARY

In some embodiments, a system for genotyping a target nucleic acid in a test sample comprise a microfluidic device having the test sample and a control sample, the control sample including a wild type of the target nucleic acid; one or more image-capturing devices to acquire images of the test and control samples to provide high-resolution melt data; and one or more processors coupled to a computer-readable media and in communication with the one or more image-capturing devices. Also, the one or more processors are configured to cause the system to obtain high-resolution melt data from the test sample defining a melting curve for the target nucleic acids in the test sample; obtain high-resolution melt data from the control sample defining a melting curve for the wild type nucleic acids in the control sample; calculate derivatives of the melting curves for the test and control sample, respectively, wherein each melting curve derivative represents a negative derivative of a fluorescence emitted from a nucleic acid sample as a function of continuously ramped temperature affecting nucleic acid denaturation; calculate parameters defining differences between features of test and control sample melting curve derivatives; and assign a genotype to the test sample based on a comparison of the calculated parameters to predetermined thresholds and boundaries defining genotype.

Some embodiments of a method for genotyping a target nucleic acid in a test sample comprise providing a microfluidic device having the test sample and a control sample, the control sample including a wild type of the target nucleic acid; providing one or more image-capturing devices to acquire images of the test and control samples to provide high-resolution melt data; and providing one or more processors coupled to a computer-readable media and in communication with the one or more image-capturing devices. Also, the computer-readable media comprises instructions for obtaining high-resolution melt data from the test sample defining a melting curve for the target nucleic acids in the test sample; obtaining high-resolution melt data from the control sample defining a melting curve for the wild type nucleic acids in the control sample; calculating derivatives of the melting curves for the test and control sample, respectively, wherein each melting curve derivative represents a negative derivative of a fluorescence emitted from a nucleic acid sample as a function of continuously increasing temperature causing nucleic acid denaturation; calculating parameters defining differences between features of test and control sample melting curve derivatives; and assigning a genotype to the test sample by comparing the calculated parameters to predetermined thresholds and boundaries defining genotypes.

### BRIEF DESCRIPTION OF THE DRAWINGS

FIG. 1 illustrates an example embodiment of an automatic genotyping system.

FIG. 2 illustrates an example embodiment of the flow of information during an operational flow for high-resolution melt analysis.

FIGS. 3A and 3B illustrate an example embodiment of a negative derivative of a heterozygous (HET) sample melting curve with an example embodiment of a Van't Hoff mixture-model fitting result.

FIG. 4 illustrates an example embodiment of a negative derivative of a heterozygous (HET) sample melting curve with example embodiments of sets of initial model parameters for a Van't Hoff mixture-model fitting.

FIG. 5 illustrates the results of an example embodiment of an expectation maximization process that used a set of initial model parameters for a Van't Hoff mixture model fitting.

FIG. 6 illustrates examples of the results of a recursive performance of an expectation maximization process.

FIG. 7 illustrates example embodiments of background-corrected negative derivative curves for a homozygous sample and a heterozygous sample.

FIG. 8A illustrates a left-sided negative-derivative-curve difference between an embodiment of a heterozygous sample and an embodiment of a wild-type control sample.

FIG. 8B illustrates a left-sided negative-derivative-curve difference between an embodiment of a homozygous or wild-type sample and an embodiment of a wild-type control sample.

FIG. 9 illustrates an example embodiment of an operational flow for the computation of the genotype probability a sample.

FIG. 10 illustrates an example embodiment of an operational flow for processing a set of melting curves of samples and ultimately determining the genotype of each tested sample.

FIG. 11 illustrates an example embodiment of an operational flow for assigning a genotype to a sample.

FIG. 12A illustrates an example embodiment of a left-sided negative-derivative-curve comparison between a wild-type control sample and a tested sample whose genotype needs to be determined.

FIGS. 12B-C illustrate example embodiments of left-sided and right-sided curve comparisons between the background-corrected negative derivative curves of a wild-type control sample and unknown samples.

FIGS. 12D-E illustrate example embodiments of curve features that may be used to determine the genotype of a sample.

FIG. 13 illustrates example embodiments of statistical measures that may be used as criteria for genotyping a sample.

FIG. 14 illustrates an example embodiment of a configuration file.

FIG. 15 illustrates an example embodiment of an operational flow for Expectation Maximization.

FIG. 16A illustrates an example embodiment of an original negative derivative curve, a background reaction curve, a residual background curve, and a reaction model curve.

FIG. 16B illustrates an example embodiment of a temperature range.

FIG. 17A illustrates an example embodiment of an original negative derivative curve, a background reaction curve, a residual background curve, and a reaction model curve.

FIG. 17B illustrates an example embodiment of a comparison between background-corrected negative derivative curves of a wild-type control sample and a tested unknown sample.

FIG. 17C illustrates an example embodiment of temperature boundaries as a basis for a genotyping decision.

FIG. 18A illustrates an example embodiment of an original negative derivative curve, a background reaction curve, a residual background curve, a first reaction model curve, and a second reaction model curve.

FIG. 18B illustrates an example embodiment of a comparison of a wild-type control sample's negative derivative curve and a tested unknown sample's background-corrected negative derivative curve.

FIG. 18C illustrates example embodiments of temperature boundaries.

FIG. 19 illustrates an example embodiment of an automatic genotyping system.

FIG. 20 illustrates an example embodiment of an operational flow for assigning a genotype to a sample.

### DESCRIPTION

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

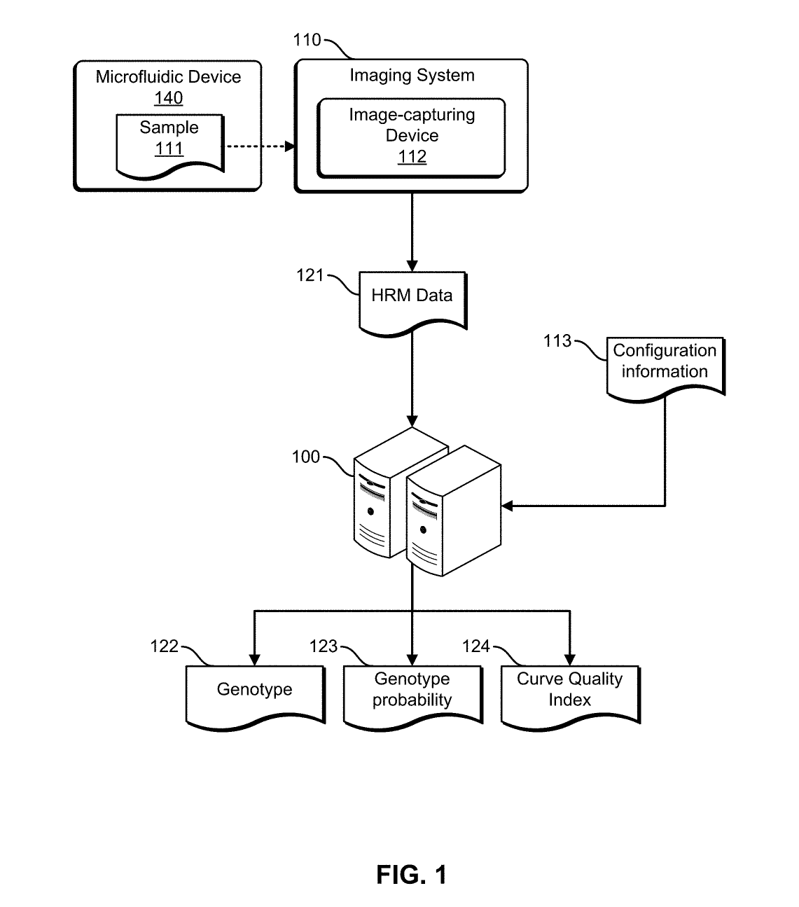

FIG. 1 illustrates an example embodiment of an automatic genotyping system. The system includes one or more genotyping devices 100 and an imaging system 110. The imaging system 110, which includes an image-capturing device 112, obtains high-resolution melt (HRM) data 121 from the high-resolution melt of a DNA sample 111. The high-resolution melt of the DNA sample 111 is performed by a microfluidic device 140. The HRM data 121 is generated from a fluorescent signal that is emitted by the sample 111 as the temperature of the sample 111 is increased by the microfluidic device 140, and the HRM data 121 defines the respective melting curve of the sample 111. The sample 111 may be included in a product that includes primers and dye for the PCR.

The genotyping devices 100 obtain the HRM data 121 from the imaging system 110, and the genotyping devices 100 obtain configuration information 113 from one or more input devices or other computing devices. The configuration information 113 may be specific for an assay and may be formatted as a configuration file. The configuration information 113 may include one or more of the following: the temperature or fluorescence range for the curve analysis, an indication whether an internal temperature control (ITC) is present in the considered assay, curve smoothing and derivative parameters, and the parameters for a Van't Hoff mixture model fitting.

The genotyping devices 100 determine a genotype 122 of the sample 111 based on the sample's HRM data 121 and on the configuration information 113. The genotyping devices 100 may also generate a genotype probability 123 and a melting curve quality index 124 (CQI) based on the HRM data 121 and the configuration information 113.

Thus, the automatic genotyping system automatically determines the genotype of unknown samples based on their melting-curve features. Because the system uses some a priori information (such as a control sample) and is based on curve differentiation between an unknown sample melting curve and one or more control sample melting curves, some embodiments of the system check the relevance and quality of these control sample melting curves prior to performing analysis and genotype determination on any unknown sample melting curve.

In some embodiments, the system performs the same basic operations on all of the sample melting curves (e.g., control sample melting curves, unknown sample melting curves). These operations can include curve pre-processing and Van't Hoff mixture model (MM) fitting. During the MM fitting operation, initialization differs depending upon the a priori nature type of the samples (e.g., wild-type (WT) control sample, non-template control (NTC) sample, and an unknown sample). Likewise, the final decision-making operation can be split into different decision-making processes depending on the a priori type of the tested samples.

For example, some embodiments of the automatic genotyping system require only one melting curve of a WT sample to serve as a negative control for a pair-wise curve comparison and genotype determination of any unknown sample, and these embodiments operate without any manual input during the comparison and determination.

Also, the automatic genotyping system determines whether a sample's melting curve reveals features of a target mutation or a known non-target mutation (either homozygous or heterozygous mutations) for the considered assay that is being tested. In some embodiments, the automatic genotyping system labels the genotype mutation as ‘Present’, ‘Absent’, ‘No-call,’ or ‘Invalid Test’. Theses labels are defined as follows: Result ‘present’: the unknown sample's melting curve reveals significant features of the target homozygous or heterozygous mutation for the considered assay. Result ‘absent’: the sample's melting curve does not reveal features of the target mutation. Result ‘no-call’: the sample's melting curve reveals features that are neither those of the target mutation for the considered assay or other known non-target mutations. Result ‘invalid’: the sample's melting curve, the WT control's melting curve, or the NTC melting curves are of insufficient quality or invalid.

The automatic genotyping system can analyze each sample independently and can follow a defined computing order. In some embodiments, the WT control sample is analyzed before any unknown sample is analyzed because the WT control sample may be used for a pair-wise curve comparison and genotyping determination on the unknown sample. Additionally, the automatic genotyping system may use a priori information, parameters, or thresholds, all of which can be included in the configuration information 113. The a priori information, parameters, and thresholds can be derived theoretically or using an independent training set of DNA sample melting curves for the considered assay.

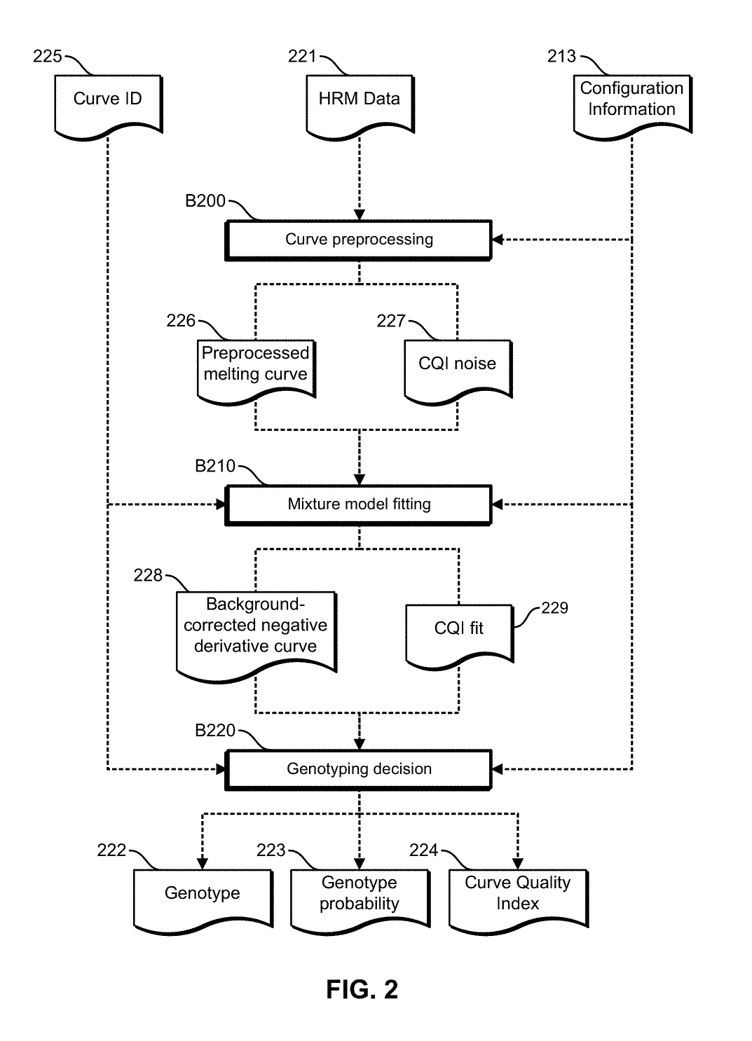

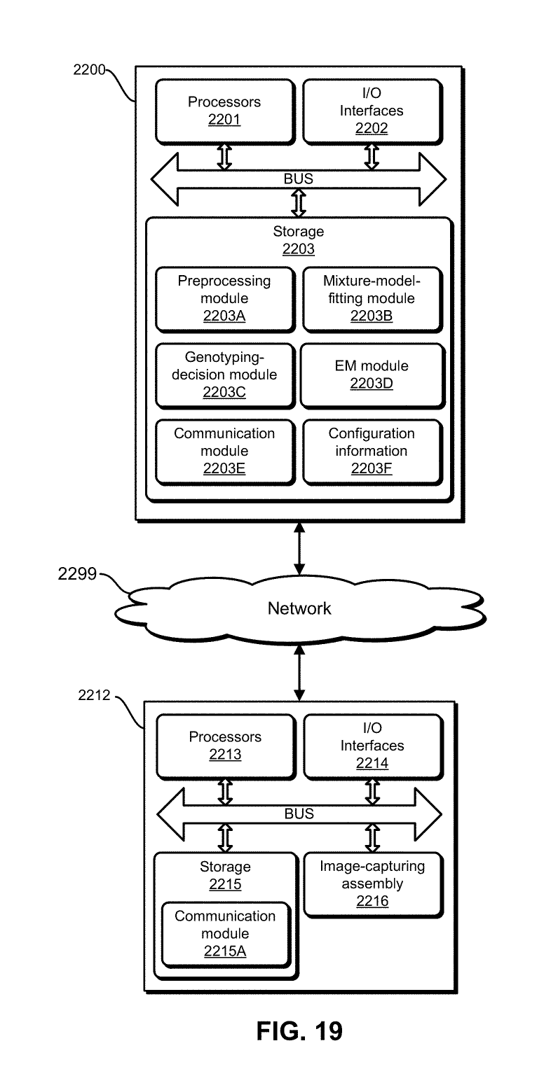

FIG. 2 illustrates an example embodiment of the flow of information during an operational flow for high-resolution melt analysis. The blocks of this operational flow and the other operational flows that are described herein may be performed by one or more devices, for example the devices and systems that are described herein (e.g., the automatic genotyping system in FIG. 1, the automatic genotyping system in FIG. 19). Also, although the operational flows that are described herein are each presented in a certain computing order, some embodiments may perform at least some of the operations in different orders than the presented orders. Examples of possible different orderings include concurrent, overlapping, reordered, simultaneous, incremental, and interleaved orderings. Thus, other embodiments of the operational flows that are described herein may omit blocks, add blocks, change the order of the blocks, combine blocks, or divide blocks into more blocks.

First, an overview of the operational flow will be presented, and then a more detailed explanation will be presented.

In block B200, preprocessing is performed based on HRM data 221, which defines one or more melting curves, and on configuration information 213, thereby generating one or more preprocessed melting curves 226, such as the negative derivative curves of the melting curves that are defined by the HRM data 221. Also, curve quality index (CQI) noise 227 is computed based on the HRM data 221.

After block B200, the flow moves to block B210, where the curve identification (ID) 225 is obtained. The curve ID 225 may be included in the configuration information 213. The curve ID 225 indicates if the DNA samples are control (CTRL) samples, non-template control (NTC) samples, or unknown (genotype to be determined by the device) samples. The curve ID 225 may be entered prior to obtaining the high-resolution melt (HRM) data 221 and may be included in the configuration information 213 for specified data processing and decision mechanics that depend upon the sample being analyzed. Additionally, in block B210 mixture model (MM) fitting is performed on the one or more preprocessed melting curves 226, such as the sample negative derivative curves of the melting curves, the fit portion of the CQI is measured, and the background reaction curves are subtracted from the sample negative derivative curves. In some embodiments, to reduce the computational time, the MM fitting is performed only on a region-of-interest of a sample negative derivative curve instead of the entire curve. This region-of-interest may be a limited temperature range where sample genotyping is depicted and may be fully defined in the configuration information 213 for the assay that is being tested. Block B210 outputs the one or more background-corrected negative derivative curves 228, which have had their background reaction curves removed, and outputs the measured CQI fit 229, which is indicative of the goodness of the model fit or tightness of the model fit with the sample curve.

Finally, in block B220, if the sample being analyzed is a control sample, such as a wild-type (WT) CTRL sample or an NTC sample, then, based on the configuration information 213, on the one or more background-corrected negative derivative curves 228, and on the CQI fit 229, the sample background-corrected negative derivative curve is checked to determine if it has expected features. If the sample being analyzed is an unknown sample, then a genotype 222 is determined for the sample using the configuration information 213, on the one or more background-corrected negative derivative curves 228, and on the CQI fit 229. Also a genotype probability 223 and an overall CQI 224 are calculated. The overall CQI 224 for a melting curve may be the square root of the product of the CQI noise 227 and the CQI fit 229, for example.

The operations in block B200, B210, and B220 are described in more detail below.

In some embodiments, the preprocessing in block B200 includes the following: resampling a melting curve to an equally-spaced temperature scale using the average rate of consecutive temperature points of the original melting curve as the rate of the resampled melting curve, removing some of the noise present in the melting curve through data smoothing, and computing the negative derivative for each melting curve. The negative derivative curve is obtained using the melting curve, and the negative derivative curve presents information on the sample melt in a different manner: the local slope of the melting curve (the sample fluorescence), −dF/dT (where F is sample fluorescence and T is temperature), is presented as function of the temperature. Smoothing and negative derivatives may be estimated using the Savitzky-Golay (SG) filter with a polynomial degree of 2. Also, an iterative smoothing can be used for noise reduction. A temperature window size and a number of iterations can be predefined through preliminary investigation of an assay using an independent set of training samples, and these parameters can be included in the configuration information 213.

Also, in some embodiments of block B200, the CQI noise 227, which is an initial measure of a sample curve quality, can be described according to the following:

CQIₙₒᵢₛₑ=100(1−qₙₒᵢₛₑσ),

where σ is the standard deviation or a median of absolute deviations, for example, of the difference between the original melting curve and the smoothed melting curve, and where qₙₒᵢₛₑ is a scaling constant. In some embodiments, the CQI noise 227 will be greater than or equal to 0—the CQI noise 227 is set to zero if the computed CQI noise 227 is less than 0. This embodiment of the CQI noise 227 indicates the degree (or percent) of noise in the original HRM data 221. Other measurements of the noise may be used for the sample CQI noise 227.

The MM fitting in block B210 may help identify features of each individual reaction that is ongoing during the high-resolution melt of a product that includes a sample. In some embodiments, the basis of each reaction is described by using a Van't Hoff mixture model, and each reaction is assumed to be independent of the other reactions in the high-resolution melt of a product. Background reactions that are caused by remaining unused primers or dye for a PCR reaction or a temperature dependence on the intercalated dye can also be modeled as a single reaction using the Van't Hoff mixture model. The resulting product melting profile is modeled as the weighted sum of independent reactions, each of which is described by a respective reaction model. Thus, from the original HRM data or the original negative derivative curve, the MM fitting generates a respective reaction model for each reaction that occurs during the high-resolution melt of the product that includes the sample.

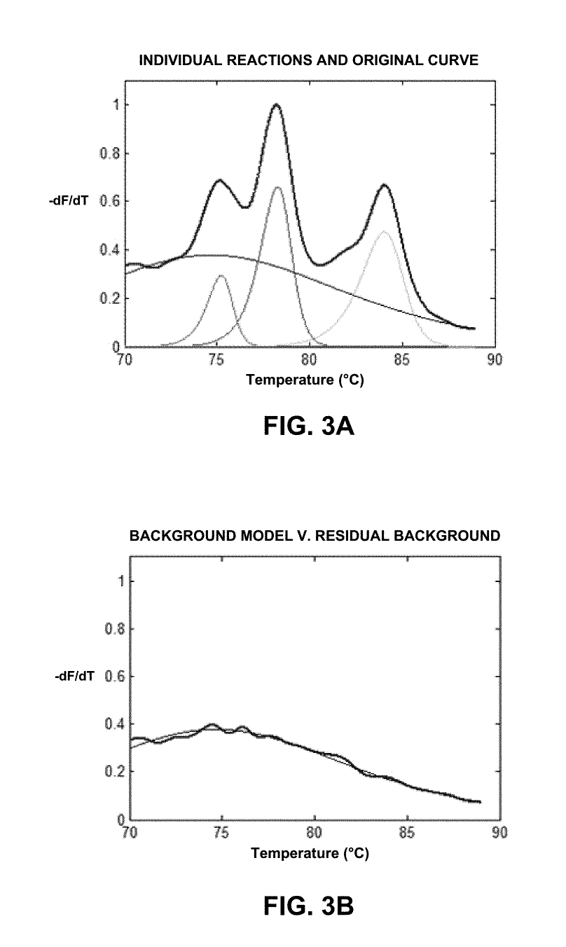

FIGS. 3A and 3B illustrate an example embodiment of a negative derivative of a heterozygous (HET) sample melting curve with an example embodiment of a Van't Hoff mixture model fitting result. The sample is being tested for a prothrombin G20210A mutation (or factor II mutation). FIG. 3A shows the negative derivative of the sample melting curve (i.e., the negative derivative curve) and the individual reaction models (two for the amplicons; one for the internal temperature control (ITC), which is a synthetic product added for more accurate control and measurement of the temperature; and one for the background reaction). A reaction model is a model of a single reaction (e.g., an amplicon reaction, an ITC reaction, a background reaction), and a melting curve is composed of one or more reactions and can be modeled as a mixture of reaction models. The models can be applied to the fluorescence or the negative derivative of the fluorescence. The background reaction model (“background model”) is a Van't Hoff model curve of the background reaction (the non-DNA reaction). The residual sample background is the difference between the measured melting curve and the reaction model components that compose the DNA reactions. This residual sample background should look very similar to the background model if the overall modeling is good.

FIG. 3B shows the resulting MM background reaction curve and shows the sample residual background curve, which is obtained by subtracting all non-background reaction model curves from the sample negative derivative curve. The standard deviation between the background reaction curve and the sample residual background curve is measured and is used to select the initial set of model parameters that best apply to the underlying sample genotype and that achieve the smallest standard deviation measure.

To determine each reaction's weight and features (e.g., enthalpy change and melting temperature) during an MM fitting, Expectation Maximization (EM) may be used. EM is an iterative process that may be initiated using a set of model parameter values, which may be obtained through a rough estimation step or using a priori information on the relative reaction features of different genotype samples for the assay being tested, and that, by means of a gradient descendent-type process, re-estimates the parameters until convergence to a solution is reached.

Selection of the initial model parameters for features of each reaction involved in a considered sample facilitates the convergence of the EM toward the global optima. A set of initial parameters that is chosen relatively far-away from the solution may not allow convergence to the global optimal, but may instead converge to a local remote minimum.

To ensure successful and rapid convergence of the EM, the selection of the initial parameters for each reaction can be implemented so that multiple sets of starting reaction parameters are tried out. The selection may also rely on the type of assay being analyzed and on the type of the sample being analyzed (e.g., a WT CTRL sample, an NTC sample, or an unknown sample). A priori information on the features of the sample can also be used. Initial parameters (e.g., melting temperature and enthalpy change) may be contained in or derived from the configuration information 213. In some embodiments, each initial set of parameters is individually input to the EM for a limited number of iterations. The standard deviation between the background reaction curve and the sample residual background curve (i.e., the curve resulting from subtraction of all the reaction model components that compose the DNA reactions from the original negative derivative curve) is then calculated. The set of initial parameters that achieve a minimal standard deviation can be retained as the best set, and the EM is resumed using that set.

The initial model parameters are set for each potential reaction, which depict features of all possible known genotypes, and for any added synthetic product, such as an internal temperature control (ITC) product. An ITC product may be used for small-amplicon-assay testing and may increase the precision in temperature measurement and control, thereby increasing the distinction between genotypes. As an example using the prothrombin G20210A mutation assay (or factor II assay), and depending on the type of the sample to be analyzed, some embodiments of the initial model parameters selection implement the following:

For a WT CTRL sample, there may be three distinct reactions (one reaction that is relative to the DNA sample itself, one reaction for the ITC, and one reaction for the background) to be considered in the mixture model. A priori information on the melting temperature points of a WT CTRL sample and an ITC product are read from the configuration information 213. To account for possible variations across instruments, a couple of sets of possible initial parameters for the reactions are established by varying the values described in the configuration information 213 (e.g., by adding or subtracting 1 (up to 2° C.) from the values contained in the configuration information 213). Each individual set is then input to the EM process for a small number of iterations, and the standard deviation of the difference between the background reaction curve and the sample residual background curve is calculated. After trying out each set of initial parameters through the EM process, the set of initial parameters that leads to a minimal standard deviation measure is retained, and the EM process is subsequently resumed using that set for additional iterations.

For an unknown sample, potential initial parameters for each known genotype (e.g., WT, HOM, and HET for the prothrombin G20210A mutation assay) or none (e.g., NTC) are tested. This means that, for each potential genotype, multiple reactions and their features are tested.

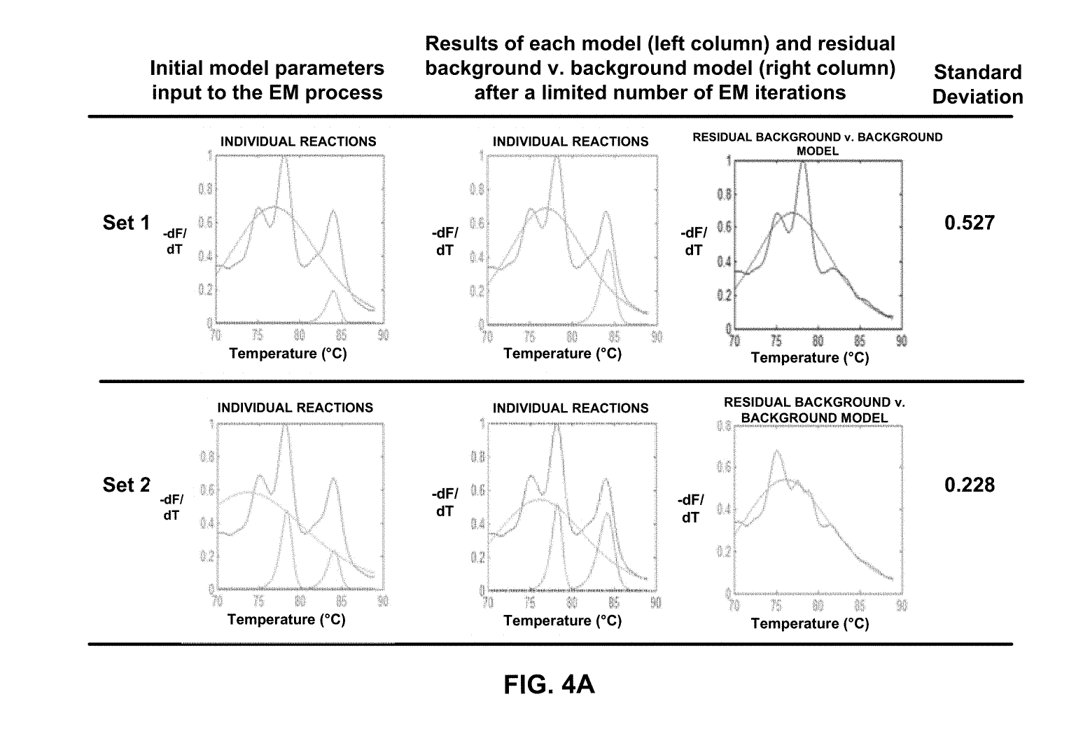

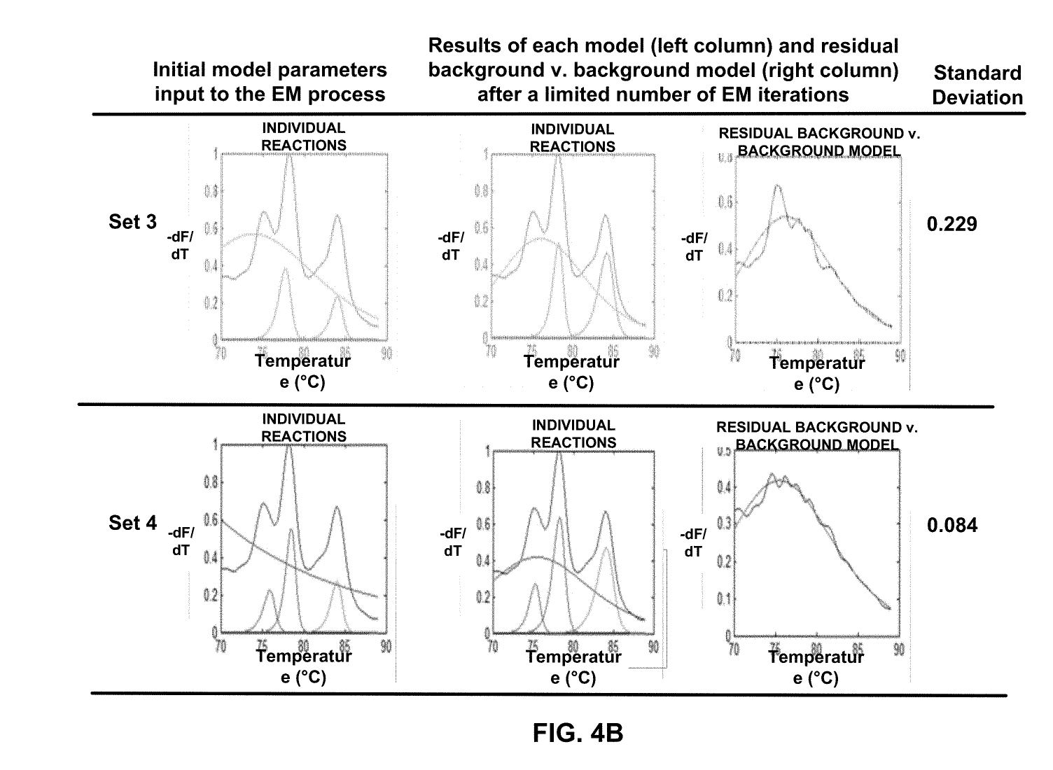

For example, for the HET for prothrom bin G20210A mutation testing (or factor II mutation testing) that is illustrated in FIG. 3A, in that set there are four reactions to account for (two for the amplicon, one for the ITC, and one for the background reaction). Because the number of reactions is unknown, the features of all genotypes are tested. Relative parameter values of each set are established using the reaction model of the WT control sample (a sample that can be processed first) and values contained in the configuration information 213. A total of four sets of initial model parameters are tested, as shown in FIG. 4. Set 1 is ITC, set 2 is WT+ITC, set 3 is HOM+ITC, and set 4 is HET+ITC. The sets are used in a limited number of iterations of the EM process, thereby generating their respective resulting standard deviations of the difference between the background reaction curve and the sample residual background curve. In this example, set 4 achieves the lowest standard deviation, and only set 4 is retained because set 4 is the set that achieves the lowest standard deviation. In some embodiments, the standard deviation (error) is weighted according to the number of reaction models to account for the fact that more model components will typically have the ability to reduce the error further by essentially modeling significant noise components. Thus the errors using four reactions may need to be significantly better than that obtained by using three reactions before a declaration of a set using four reactions is made (HET+ITC).

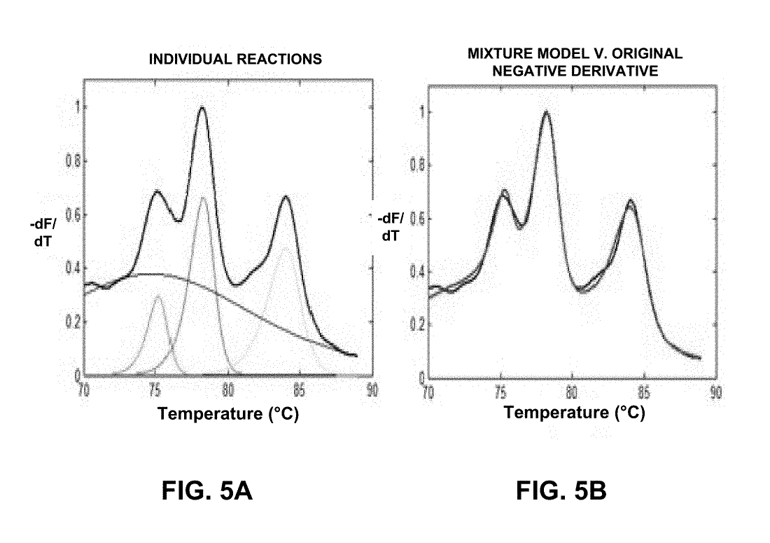

The EM process is continued with additional iterations using the set that achieves the lowest standard deviation until convergence is reached. FIG. 5 illustrates the results of an example embodiment of an expectation maximization process that used a set of initial model parameters for a Van't Hoff mixture model fitting. The set of initial model parameters was the set in FIG. 4 that achieved the smaller standard deviation, which was set 4. In FIG. 5, the left graph shows the original negative derivative curve of the melting curve and the individual resulting reaction models. The right graph shows the mixture model results overlapped with the original negative derivative.

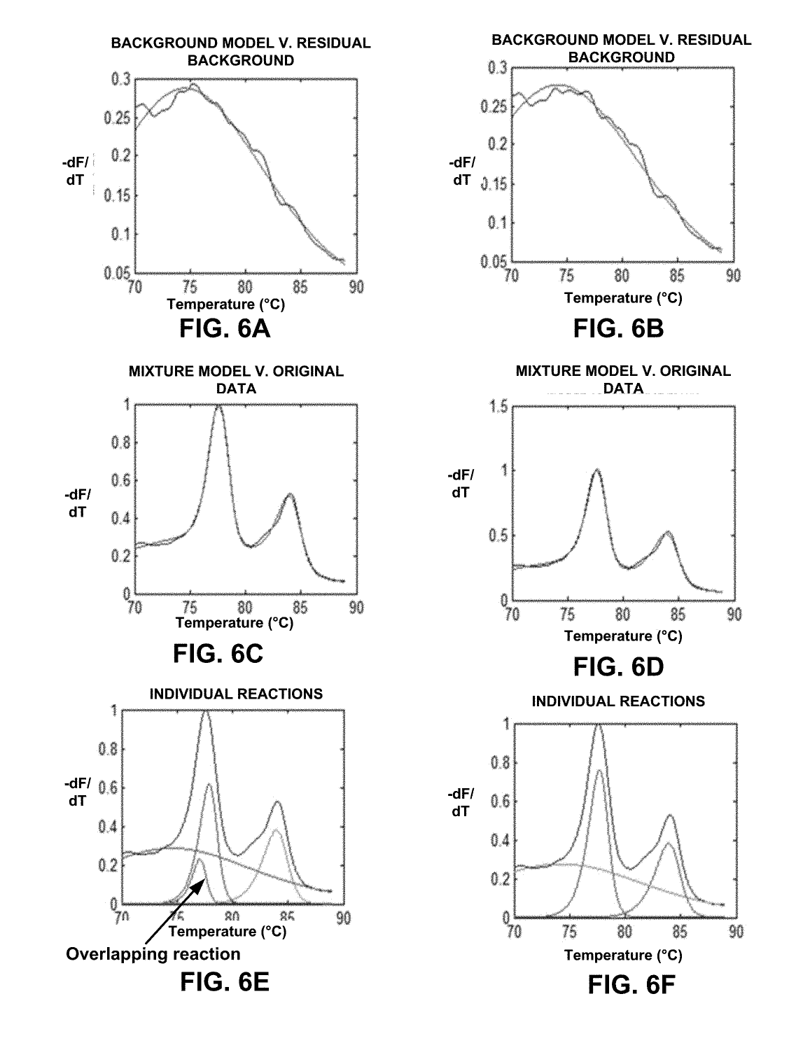

After the EM process is completed, the results may be input to a post-processing operation in which the overlaps between pairs of reaction models, not including the background reaction, are inspected. When two reactions overlap more than a threshold (e.g., 95%), some embodiments then discard one of the two reactions from the mixture model and perform additional iterations of the EM process to account for the removal of the reaction from the mixture model. Examples of the results of a recursive performance of an EM process are illustrated in FIG. 6.

In FIG. 6, the graphs in the left column show the results of the EM process, which was resumed using an initial set of four reactions (two for the amplicon, one for the ITC, and one for the background). The graphs in the left column, particularly the graph in the bottom left, show that one of the mixture reaction models fully overlaps another reaction. The overlapping reaction is discarded from the mixture model by some embodiments. The graphs in the right column show the final mixture model, which was obtained by resuming the EM process for additional iterations on a set of reactions that do not include the discarded reaction.

After the MM fitting, a goodness-of-fit measure may be derived by comparing the difference of the sample negative derivative curve to the resulting mixture model. The goodness-of-fit measure may be a height of the curve difference (maximum of the curve difference minus the minimum of the curve difference). This measure provides, to some extent, information on the waviness of the data compared to the mixture model. Small wavy patterns are commonly observed on the negative derivative curves and may not affect the correct genotyping of the samples. However, such a wavy pattern, if more pronounced, may affect a genotyping decision and may be due to product contamination. Therefore, measuring the difference between the mixture model and the original negative derivative curve provides information on both the quality of the data acquired by a system, the quality of the assay, and the goodness of the model fit. Also, for example, if the sample negative derivative curve presents an unusual bump that is not included in the mixture model's curve, then the difference between the mixture model and the sample negative derivative curve will be relatively large and can be accounted for by conveying the detected issue or poor quality through the Curve Quality Index (CQI) fit 229.

Similar to the CQI noise 227, the CQI fit 229 may be expressed in a percent, for example as follows:

CQI_{fit}=100(1−q_{fit}h),

where h is the height of the difference between the sample negative derivative curve and the resulting mixture model curve, and where q_{fit} is a scaling constant. In some embodiments, the CQI fit 229 must be greater than or equal to zero (CQI_{fit} is set to zero if it is less than zero). Other measures of curve fit may also be used, such as KL-divergence based methods, median absolute deviation of the residual, and mean squared error.

After completion of the EM process, features of each underlying reaction, including features of the background reaction, are fully defined. Sample genotyping may be performed at this stage without any additional data processing. To ensure robust and accurate determination of the sample genotype, some embodiments further derive a background-corrected negative derivative curve 228, which is a melting curve where the background reaction curve is removed. Such a removal (or background correction) may be performed by subtracting the background reaction's curve from the sample negative derivative curve in the HRM data 221.

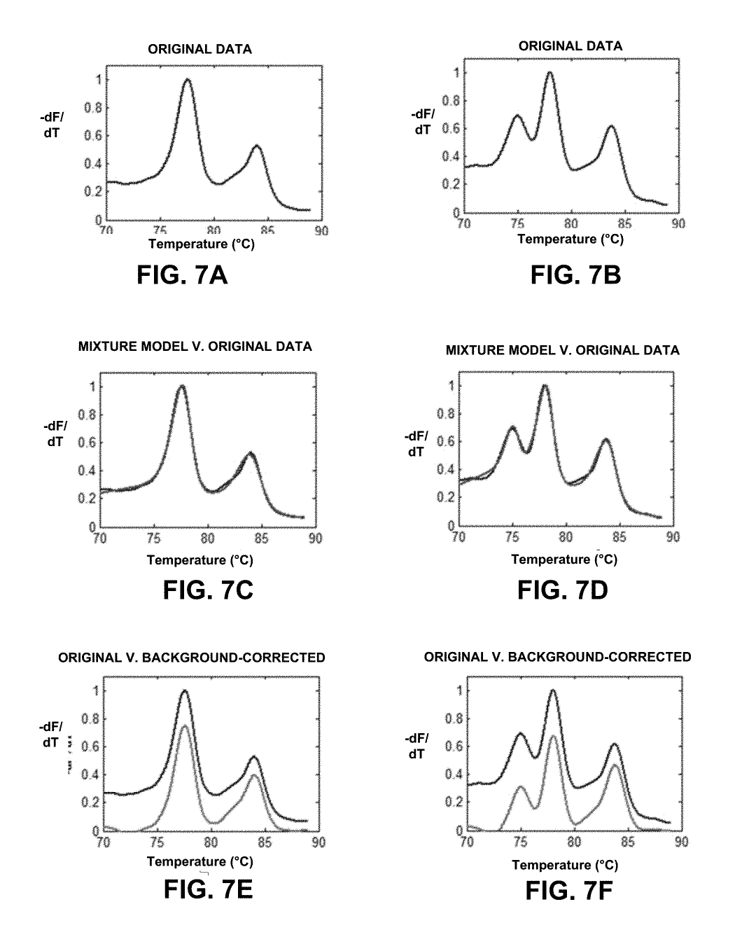

FIG. 7 illustrates example embodiments of background-corrected negative derivative curves for a homozygous sample and a heterozygous sample. The HOM sample's curves are shown in the left column, and the HET sample's curves are shown in the right column. The top two graphs show the original sample negative derivative curves. The middle two graphs show the mixture models that were generated by the EM process and show the original sample negative derivative curves. The bottom two graphs illustrate the sample negative derivative curves and the background-corrected negative derivative curves.

The genotyping decision in block B220 applies or verifies a set of basic criteria (e.g., thresholds) based on the temperature and fluorescence features of the background-corrected negative derivative curve 228 of the sample (However, some embodiments use the original negative derivative curve instead of the background-corrected negative derivative curve 228). Temperature and fluorescence features of the background-corrected negative derivative curve 228 may be defined as the point where the fluorescence reaches some local maxima. The estimation of these features may be performed using each resulting reaction of the mixture model. When the background-corrected negative derivative curve 228 is used, the local maxima of the reactions may have shifted from their original position due to subtraction of the background reaction curve. To account for this as well as any temperature variability that may be produced from the HRM data acquisition system (e.g., the microfluidic device, the imaging system), a search within a range (e.g., ±0.5° C.) of the estimates from the mixture model may be used.

The goal of block B220 may depend on the type of the sample, which may be a WT control sample, an NTC sample, and an unknown sample. In some embodiments, multiple samples are evaluated, and a WT control sample is the first sample to have its quality and validity assessed. Also, in some embodiments, NTC samples are assessed only to determine whether contamination of the assay has occurred or not. Finally, the genotyping decision on the unknown samples may include verifying their quality and determining a genotype 222 with an associated genotype probability 223 (e.g., a confidence level of the determined genotype for the given sample). In block B220, the unknown sample negative derivative curves can first be differentiated with the WT CTRL negative derivative curve. Table 1 provides a synopsis of the response when the negative derivative curve of the sample either passes or fails the criteria based on the comparison with the negative derivative curve of the WT CTRL sample. NA is an abbreviation of “Not Assigned,” and NC is an abbreviation of “No Call.” Details of the criteria used to determine the responses are described below.

TABLE 1

| | WT Control | NTC | Sample 1 | . . . | Sample N |
|---|---|---|---|---|---|
| Response | FAIL | FAIL | FAIL | | FAIL |
| | 1. Assign all | 1. Assign NA | 1. Assign NA | | 1. Assign NA |
| | samples to NA | to that sample | to sample 1; | | to sample N; |
| | (or NC); and | and all other | and | | and |
| | 2. Stop. | samples; and | 2. Continue | | 2. Stop. |
| | | 2. Stop. | with next | | |
| | | | sample. | | |
| | PASS | PASS | PASS | | PASS |
| | 1. Assign | 1. Assign NTC | 1. Assign | | 1. Assign |
| | CTRL to that | to that sample; | genotype and | | genotype and |
| | sample; and | and | associated | | associated |
| | 2. Continue | 2. Continue | probability to | | probability to |
| | with the next | with the next | sample 1; and | | sample N; and |
| | sample. | sample. | 2. Continue | | 2. Stop. |
| | | | with the next | | |
| | | | sample. | | |

In Table 1, the decision procedure depends upon a priori information on the type of the DNA sample and upon a comparison with the WT CTRL sample. Note that, in some embodiments, samples 1 through N, whose genotypes are to be determined, depend on the WT CTRL sample being processed first, but these unknown samples do not depend on each other. Additionally, the WT CTRL processing and NTC processing may have no dependencies. Thus, some operations may be performed in parallel.

The set of criteria applicable to WT CTRL samples may be used to determine the quality and validity of the WT CTRL samples. The criteria may depend on whether an ITC was added to the product for better accuracy and control of the temperature measurement. The ITC information, as well as decision thresholds on the genotype, can be included in the configuration information 213 that is specific to the considered assay. Table 2 lists an example of the sample features and the criteria for the decision making on WT control samples. The subscript “confg” indicate that the value is contained in the configuration information 213.

TABLE 2

| | | | Pass/ | |
|---|---|---|---|---|
| Criteria | | | Fail | Response |
| 1 | | CQI ≧ | FAIL | A. Assign NA to all samples, and |
| | | CQI_{confg} | | B. Stop. |
| | | | PASS | A. Valid WT CTRL sample; assign |
| | | | | CTRL to the sample, and |
| | | | | B. Go to 2. |
| 2 | w/ ITC | T_ITC within | FAIL | A. Assign NA to all samples, and |
| | | confg range | | B. Stop. |
| | | and | PASS | A. Calculate the temperature |
| | | F_ITC ≧ | | difference as follows: |
| | | F_ITC_{confg} | | ΔT = \|(Tₛₐₘₚₗₑ − T_{ITC}) − |
| | | | | (T_{CTRL}_{_{confg}} − T_{ITC}_{_{confg}})\|, |
| | | | | and |
| | | | | B. Go to 3. |
| 3 | w/o | | | A. Calculate the temperature |
| | ITC | | | difference as follows: |
| | | | | ΔT = \|Tₛₐₘₚₗₑ − T_{CTRL}_{_{confg}}\|, and |
| | | | | B. Go to 3. |
| | | ΔT ≦ ΔT_{confg} | FAIL | A. Assign NA to all samples, and |
| | | and | | B. Stop. |
| | | F ≧ F_{confg} | PASS | A. Valid WT CTRL; assign CTRL |
| | | | | to the sample, and |
| | | | | B. Continue to the next sample. |

(*The temperature of the sample Tsample and the fluorescence F are estimated based on the negative derivative curve using each reaction resulting from the mixture model).

Additionally, other checks may be applied to the mixture model coefficients or calculated enthalpies in order to ensure that the models described in the configuration information agree with the observed data.

The set of criteria applicable to NTC samples may be used to determine the quality of the acquisition system and determine if contamination of the sample has occurred. Like the criteria for the WT CTRL sample, the criteria depend upon whether an ITC is being used or not. The ITC information, as well as all decision thresholds on the sample, can be included in the configuration information 213 that is specific to the considered assay. Table 3 lists the features and successive criteria for the decision making for NTC samples. The subscript “confg” is used to indicate that the value is contained in the configuration information 213.

TABLE 3

| | | | Pass/ | |
|---|---|---|---|---|
| Criteria | | | Fail | Response |
| 1 | | CQI ≧ | FAIL | A. Invalid or low quality NTC; |
| | | CQI_{confg} | | assign NA to all samples, and |
| | | | | B. Stop. |
| | | | PASS | A. Go to 2. |
| 2 | w/ ITC | \|Tₛₐₘₚₗₑ − | FAIL | A. Assign NA to the sample, and |
| | | T_{NTCconfg}\| | | B. Continue to next sample, if any. |
| | | within confg | PASS | A. Valid NTC; assign NTC to the |
| | | range | | sample, and |
| | | and | | B. Continue to next sample, if any. |
| | | Fₛₐₘₚₗₑ ≧ | | |
| | | F_NTC_{confg} | | |
| | w/o | Background | FAIL | A. Assign NA to the sample, and |
| | ITC | peak ≧ any | | B. Continue to next sample, if any. |
| | | potential | PASS | A. Valid NTC; assign NTC to the |
| | | reaction peak | | sample, and |
| | | resulting from | | B. Continue to next sample, if any. |
| | | the mixture | | |
| | | model fit | | |

(*The temperature of the sample Tsample and the fluorescence F are estimated based on the negative derivative curve using each reaction resulting from the mixture model).

Similarly, other checks may be applied to the mixture model coefficients or calculated enthalpies in order to ensure that the models described in the configuration files agree with the observed data.

The set of criteria applicable to unknown samples can be used to determine the underlying genotype of these samples. An unknown sample can be one of three basic genotypes: HET, HOM, and WT for the targeted mutation. Depending on the targeted DNA mutation being analyzed and on the design of the assay, one or more off-target mutations (also called non-targeted mutations or sub-variants) may be revealed. These non-targeted mutations, if present in a sample, may be observed in the acquired negative derivative curve as having different HET and HOM genotype features than that for the targeted mutation. As an example, Hemochromatosis (HFE) mutation assays may include additional HOM and HET shape negative derivative curves for non-target mutations. Therefore some embodiments account for any known non-target mutations during the genotyping-decision process for an unknown sample. Non-target mutations that are infrequently encountered or unknown prior to creation of the configuration information 213 for a given assay may be determined to be “Not Assigned” (NA) for a genotype. However, in some embodiments, all known non-target mutation genotypes may be defined in the configuration information 213 for each given assay.

In the configuration information 213, the naming or labeling of targeted and non-target homozygous or heterozygous mutations may contain either “HET” or “HOM.” For example, if there are two HOM-type and three HET-type genotypes that have been observed on a considered assay, then the configuration information 213 for this assay could indicate that there are up to six possible genotypes. The genotypes could be named as follows: WT, HOM1, HOM2, HET1, HET2, and HET3. Although the resulting sample genotyping may be conveyed as such, some embodiments further relabel the genotyping result as ‘Present’, ‘Absent’, ‘No-call,’ or ‘Invalid Test’.

Whether there are targeted and non-targeted HET and HOM genotypes for a given assay, the decision process may rely on typical features of HET versus WT and HOM versus WT, as explained below:

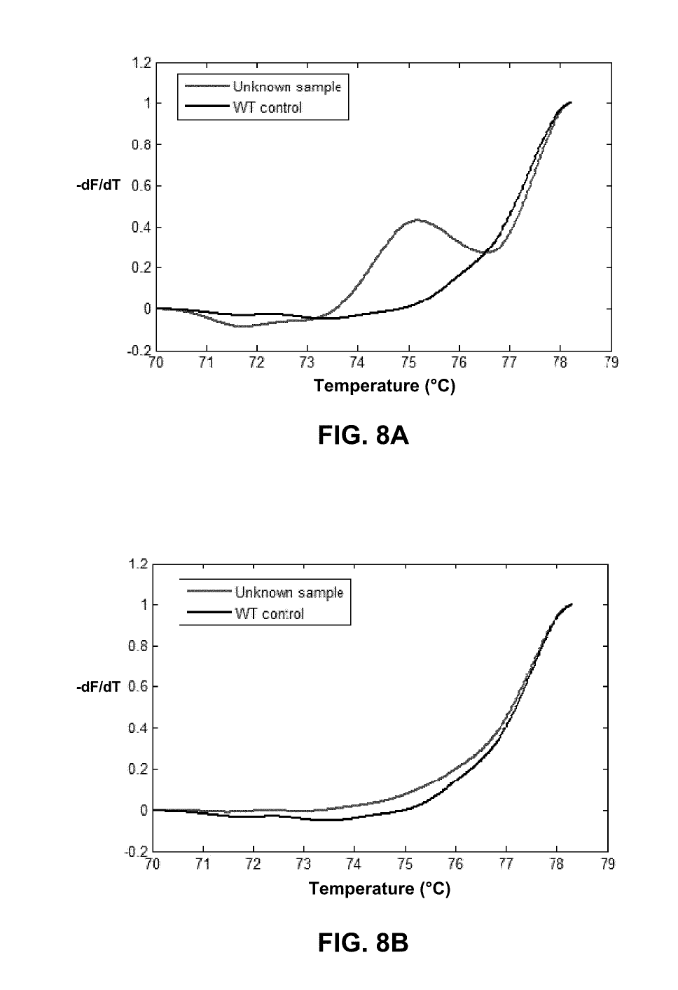

HET samples present two nearby distinct peaks on the negative derivative curve for the amplicon site as compared to only one peak for the negative derivative curve of a WT control sample. Because detection of two peaks on the negative derivative curve for a HET sample may fail in some situations, a difference of either the left or right side of the sample's melting curve may be performed against the WT control sample's negative derivative curve, after alignment and rescaling of the major peak. The determination whether to perform a left-sided curve difference or a right-sided curve difference is made with the result of the MM fit process. Each reaction resulting from the MM fit is compared to the WT CTRL sample. The reaction model which has a maximum fluorescence that is located at a temperature point nearby the WT CTRL melting temperature is labeled as the major peak. The location of the secondary peak with respect to the major peak indicates the side of the curves to be used for the curve difference. This curve difference permits identification of the HET samples from among other genotypes. FIG. 8A illustrates a left-sided negative-derivative-curve difference between an embodiment of a HET sample and an embodiment of a WT CTRL sample, and FIG. 8B illustrates a left-sided negative−derivative-curve difference between an embodiment of a HOM or WT sample and an embodiment of a WT CTRL sample. As illustrated in FIGS. 8A-8B, there is a significant difference in fluorescence at the site of the secondary amplicon peak for a HET sample when compared with the WT CTRL sample.

HOM samples can be revealed by a melting temperature or a temperature at the peak of the major reaction that significantly differs from that of a WT CTRL sample. The range in temperature difference can be established during the assay development, either theoretically or using a training set of samples with known genotypes.

Sample negative-derivative-curve features listed above can be used as the basis of the decision-making operation, and criteria on the relative fluorescence of the negative-derivative-curve peaks can be used to avoid over-determining some unknown samples to be HET. Some small “bumps” or “kicks” at the shoulder or toe of the negative derivative peak, which may be due other phenomena not directly related to the DNA sample, sometimes appear, and the criteria (e.g., threshold) on the amplitude may prevent the mis-determination of these curves. For example, the small pre-amplicon bump in unlabeled probes may be an artifact of that type of assay. The artifact may be added to the model for the assay, and the artifact may be removed from the negative derivative curve.

Also, any sample may be subject to contamination or reveal a variant that was not observed during the design of the assay. This situation is accounted for in the decision-making operation by assigning CNA to a tested sample whenever that sample does not satisfy any of the criteria or decision thresholds that are contained in the configuration information 213.

FIGS. 8A-8B illustrate left-sided differences between an unknown sample and a WT control sample in order to determine whether the unknown sample is a HET sample. The graph in FIG. 8A is an example of an unknown HET sample's negative derivative curve that is compared to the WT control sample's negative derivative curve. Remarkable local differences between these curves indicate the possibility of HET for the unknown sample genotype. The graph in FIG. 8B is an example of an unknown sample's negative derivative curve (the unknown sample being either a HOM or WT sample) that is compared to the WT control sample's negative derivative curve. The differences between these melting curves are relatively minimal, which eliminates the possibility of HET for the unknown sample genotype.

In some embodiments, an ITC is used in the product for better measurement accuracy and control of the temperature. If an ITC is used, then the criteria for genotyping a sample will account for the ITC. Thus any temperature measurement can be relative to the ITC melting temperature. Regardless of whether an ITC is used, all unknown samples may go through the same genotyping decision-making procedure. Table 4 describes the successive analysis and criteria in an embodiment of a genotyping decision-making procedure.

TABLE 4
Successive analysis and criteria for
decision making on an unknown sample genotype.

| | | Pass/ | |
|---|---|---|---|
| | Criteria | Fail | Response |
| 1 | CQI ≧ CQI_{confg} | FAIL | A. Invalid melting curve; assign |
| | | | NA to the subject sample, and |
| | | | B. Continue to the next sample, if |
| | | | any. |
| | | PASS | A. Go to 2 |
| 2 | ΔFMAX ≧ ΔF_{confg} | FAIL | B. Go to 3 |
| | and \|TMAX − T_{confg}\| | PASS | A. Assign HETxx to the subject |
| | within confg range | | sample, and |
| | | | B. Continue to the next sample, if |
| | | | any. |
| 3* | \|TMAX − T_{confg}\| | FAIL | A. Go to 4 |
| | outside confg range | PASS | A. Assign NA to the subject |
| | | | sample, and |
| | | | B. Continue to the next sample, |
| | | | if any. |
| 4** | \|Tₛₐₘₚₗₑ − | FAIL | A. Go to 5 |
| | T_{WT CTRL}_{_{confg}}\| | PASS | A. Assign WT to the subject |
| | within confg range | | sample, and |
| | and | | B. Continue to the next sample, |
| | | | if any. |
| | Fₛₐₘₚₗₑ ≧ F_WT_{confg} | | |
| 5** | \|Tₛₐₘₚₗₑ − T_{HOM}_{_{confg}}\| | FAIL | A. Assign NA to the subject |
| | within confg range | | sample, and |
| | and | | B. Continue to the next sample, |
| | Fₛₐₘₚₗₑ ≧ | | if any. |
| | F_HOM_{confg} | PASS | A. Assign HOMxx to the subject |
| | | | sample, and |
| | | | B. Continue to the next sample, if |
| | | | any. |

(*The temperature T and the fluorescence F can be estimated based on the background-corrected negative derivative curve using each reaction resulting from the mixture model. ΔFMAX is the difference between shifted and rescaled unknown sample and WT control melting curves. **Melting temperature estimates are based on non-shifted background-corrected negative derivatives. For HETxx and HOMxx, xx is for multiple HET or HOM genotypes based on the configuration information. The type retained and assigned (xx) to the unknown sample may be the one that best satisfies the criteria).

Similarly, other checks may be applied to the mixture model coefficients or calculated enthalpies in order to ensure that the models described in the configuration files agree with the observed data.

In addition to assigning a genotype 222 to a tested unknown sample, block B220 also generates an associated genotype probability 223. The genotype probability 223 of a sample is a means to convey the degree of certainty of the assigned genotype for the given sample 222. The genotype probability 223 of a sample is a basic measure of the distance of the sample features with respect to the boundary of the assigned genotype 222, and may be given in a percentage. Boundaries relative to each possible genotype are parameters that may be contained in the configuration information 213. These parameters may be derived either theoretically, using a priori knowledge of the variance of the acquisition device for sample melt, or using a training set of samples with a known genotype.

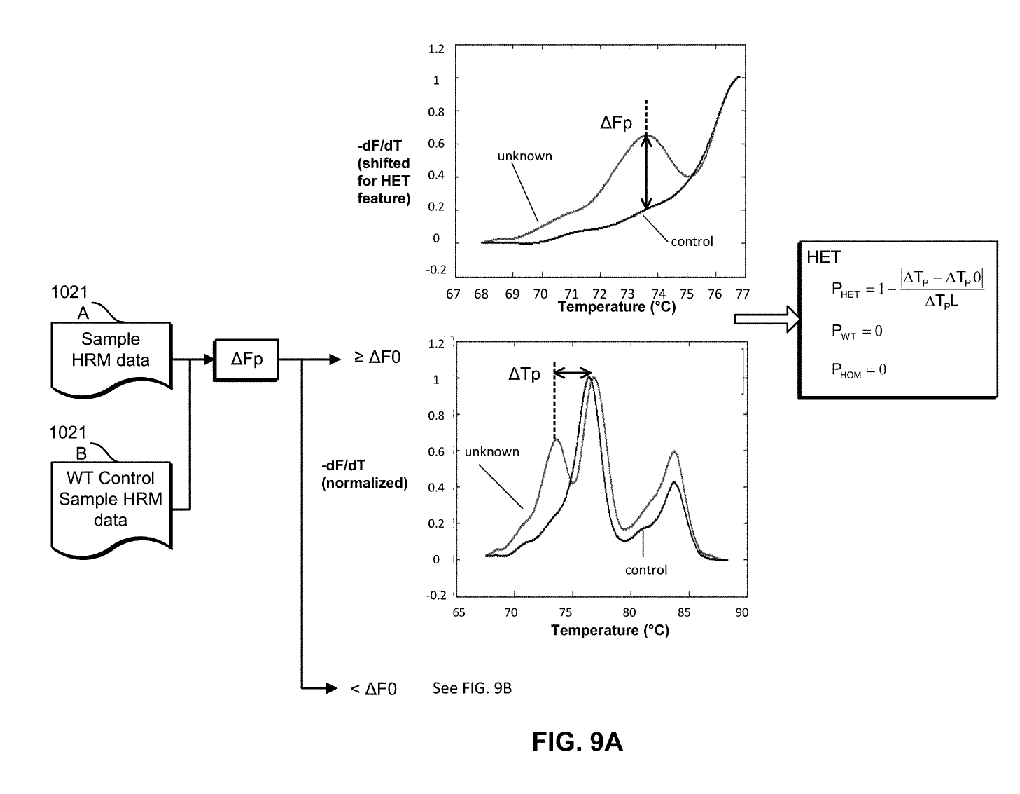

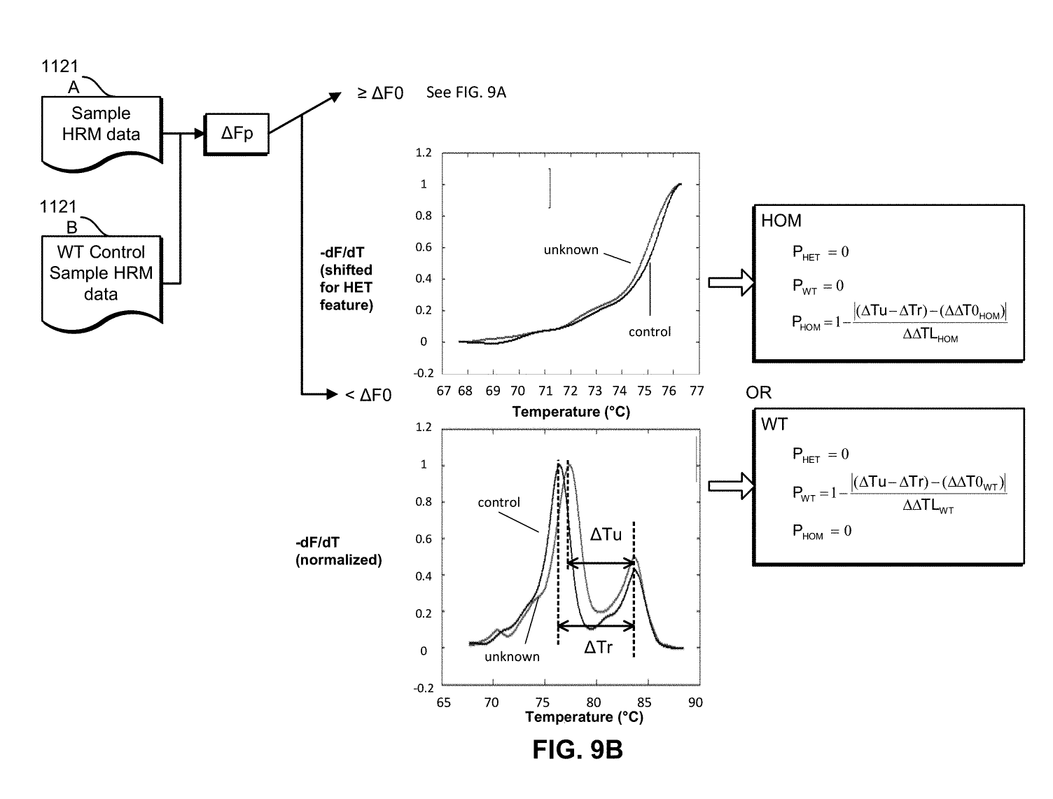

FIG. 9 illustrates an example embodiment of an operational flow for the computation of the genotype probability 223 of a sample. This example uses an assay that has three possible genotypes (i.e., WT, HET and HOM), and in which an ITC is present. Therefore, the ITC features are accounted for in providing more precise temperature measurements for the genotyping decision of a sample.

The HRM data of an unknown sample 921A and the HRM data of a WT control sample 921B are input to an operation in which the background-corrected negative derivative curves are calculated, and based on the relative distribution of the reaction models along the temperature scale, the left-side of these curves is compared. This comparison allows the determination of the maximum fluorescence difference between the two background-corrected negative derivative curves, denoted as ΔFₚ. There are two possible situations for ΔFₚ when compared to ΔF0, which is a parameter contained in the configuration information 213:
A) If ΔFₚ is greater than or equal to ΔF0, then the HET genotype is considered for the sample.
B) If ΔFₚ is less than ΔF0, then the HET genotype is eliminated for the sample, and WT or HOM are considered as a potential genotype for the sample.

Also, if ΔFₚ is greater than or equal to ΔF0, then the HET genotype is considered for the sample, and the difference between the temperature where ΔFₚ occurs and the temperature of the major reaction-model peak of the sample ΔTₚ is calculated. There are two possible situations for ΔTₚ , when compared to the defined HET boundaries contained in the configuration information 213, with ΔTₚ0 being the HET genotype center and ΔTₚL being the HET genotype range surrounding the defined center:
A) If ΔTₚ is within the defined HET boundaries, then the sample is assigned to HET, the genotype probability P of the sample being relative to HET (P _{HET}). The probability of the sample being a WT (P_{WT}) or being a HOM (P_{HOM}) are set to zero. The genotype probability of the sample may be calculated as follows:

$${P}_{HET} = 1 - \frac{\vert Δ {T}_{p} - Δ {T}_{p} 0 \vert}{Δ {T}_{p} L} .$$

B) If ΔTₚ is outside the defined HET boundaries, then the sample is considered to be a no-call or not-applicable genotype. The genotype probability P of the sample is equal to zero. In other words, P_{HET}, P_{WT}, and P_{HOM} are equal to zero.

If ΔFₚ is less than ΔF0, then the HET genotype is eliminated for the sample, and a WT genotype or a HOM genotype are considered as potential genotypes for the sample. The temperature difference ΔTᵤ between the major peak of the tested unknown sample and the ITC peak is calculated, as well as the temperature difference ΔTᵣ between the major peak of the WT control sample and the ITC peak. ΔTᵣ is further subtracted from ΔTᵤ, and the resulting value (ΔTᵤ−ΔTᵣ) is compared to WT-genotype-boundary parameters and HOM-genotype-boundary parameters, as contained in the configuration information 213. There are three possible situations:
A) If (ΔTᵤ−ΔTᵣ) is within the WT-genotype boundaries, then the sample is assigned to WT, and the genotype probability of the sample may be calculated as follows:

$${P}_{WT} = 1 - \frac{\vert ( Δ {T}_{u} - Δ {T}_{r} ) - ( ΔΔ T {0}_{WT} ) \vert}{ΔΔ {TL}_{WT}}$$

where ΔΔT0_{WT} and ΔΔTL_{WT} are parameters defining the WT-genotype boundaries (ΔΔT0_{WT} and ΔΔTL_{WT} are contained in the configuration information 213), where ΔΔT0_{WT} is the WT-genotype center, and where ΔΔTL_{WT} is the WT-genotype range surrounding the defined WT-genotype center.
B) If (ΔTᵤ−ΔTᵣ) is within the HOM-genotype boundaries, then the sample is assigned to HOM, and the genotype probability of the sample may be calculated as follow:

$${P}_{HOM} = 1 - \frac{\vert ( Δ {T}_{u} - Δ {T}_{r} ) - ( ΔΔ T {0}_{HOM} ) \vert}{ΔΔ {TL}_{HOM}} ,$$

where ΔΔT0_{HOM} and ΔΔTL_{HOM} are parameters defining the HOM-genotype boundaries (ΔΔTO_{HOM} and ΔΔTL_{HOM} are contained in the configuration information 213), where ΔΔTO_{HOM} is the HOM-genotype center, and where ΔΔTL_{HOM} is the HOM-genotype range surrounding the defined HOM-genotype center.
C) If (ΔTᵤ−ΔTᵣ) is neither within the WT-genotype boundaries or within the HOM-genotype boundaries, then the sample is genotyped as a no-call or a not-applicable. The genotype probability P of the sample is equal to zero. In other words, P_{HET}, P_{WT}, and P_{HOM} are set to zero.

In some embodiments, genotype probabilities are generated by assuming underlying distributions (e.g., Gaussians) of ΔTₚ and ΔTᵤ and calculating the probability of the genotype given the measurements.

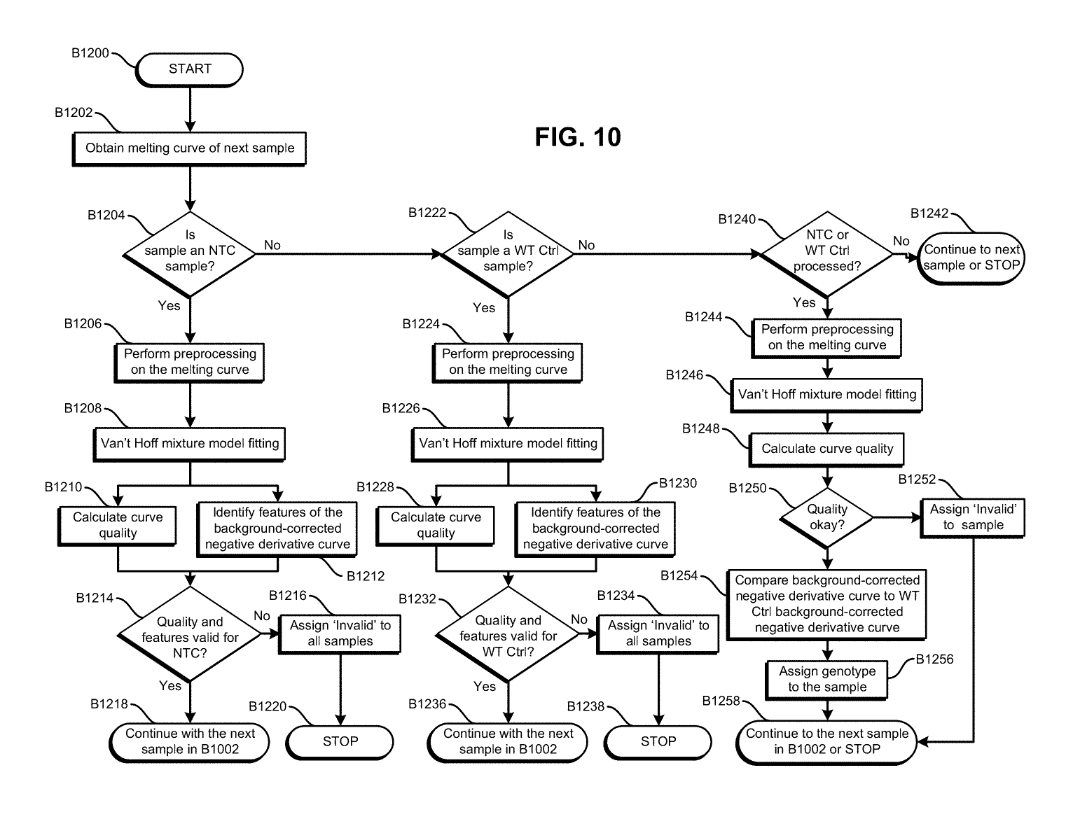

FIG. 10 illustrates an example embodiment of an operational flow for processing a set of melting curves of samples and ultimately determining the genotype of each tested sample. The operational flow is implemented by one or more genotyping devices. The flow starts in block B1000 and then proceeds to block B1002, where respective melting curves of one or more samples are obtained. Next, in block B1004, the one or more genotyping devices determine, for example based on a naming convention of samples, if the next sample is an NTC sample. If the one or more genotyping devices determine that the sample is an NTC sample, then the flow moves to block B1006.

In block B1006, the one or more genotyping devices perform preprocessing on the melting curve of the NTC sample and provide (e.g., calculate) the corresponding negative derivative curve of the sample. Then, in block B1008, the one or more genotyping devices fit the negative derivative curve to the Van't Hoff mixture model to generate a background-corrected negative derivative curve of the NTC sample. The flow then moves to block B1010, where the curve quality of the background-corrected negative derivative curve is calculated, and to block B1012, where features of the background-corrected negative derivative curve are identified. After blocks B1010 and B1012, the flow proceeds to block B1014.

In block B1014, the one or more genotyping devices determine if the curve quality and features of the background-corrected negative derivative curve are valid and representative of an NTC sample, for example by applying the criteria in Table 3. If the resulting curve quality and features are valid, then the flow moves to block B1018, where the one or more genotyping devices return to block B1002. If the resulting curve quality and features are not valid for an NTC sample, then the flow moves to block B1016, where ‘Invalid’ is assigned to all of the tested samples, and the flow moves to block B1020, where it stops.

However, if in block B1004 the one or more genotyping devices determine that the sample is not an NTC sample, then the flow moves to block B1022. Also, some embodiments of the operational flow do not include blocks B1004-B1020, so the flow proceeds directly to block B1022 from block B1002. In block B1022, the one or more genotyping devices determine if the next sample is a WT control sample, for example based on a naming convention of samples. If the one or more genotyping devices determine that the sample is a WT control sample, then the flow moves to block B1024.

In block B1024, the one or more genotyping devices perform preprocessing on the melting curve of the sample and provide the corresponding negative derivative curve of the sample. Then, in block B1026, the one or more genotyping devices fit the negative derivative curve to the Van't Hoff mixture model to generate a background-corrected negative derivative curve of the WT CTRL sample. The flow then moves to block B1028, where the curve quality of the background-corrected negative derivative curve is calculated, and to block B1030, where features of the background-corrected negative derivative curve are identified. After blocks B1028 and B1030, the flow proceeds to block B1032.

In block B1032, the one or more genotyping devices determine if the curve quality and features of the background-corrected negative derivative curve are valid and representative of a WT control sample for the assay being tested, by applying the criteria in Table 2 for example. If the curve quality and features are valid, then the background-corrected negative derivative curve and the calculated curve quality of the WT control sample are stored in storage, and then the flow moves to block B1036, where the one or more genotyping devices return to block B1002. If the curve quality and features are not valid for a WT control sample for the assay being tested, then the flow moves to block B1034, where “Invalid” is assigned to all of the samples, and then the flow moves to block B1038, where it stops.

However, if in block B1022 the one or more genotyping devices determine that the sample is not a WT control sample, then the flow moves to block B1040. In block B1040, the one or more genotyping devices determine if an NTC sample or a WT control sample have been processed. The sample may have been processed immediately before, or it may have been processed hours days, weeks, or months before) and the results stored in storage.

If an NTC sample or a WT control sample has not been processed, then the flow moves to block B1042, wherein the flow returns to block B1002 or stops. In some embodiments, the flow moves to block B1042 only if neither an NTC sample nor a WT control sample have been processed, and in some embodiments the flow moves to block B1042 if either an NTC sample or a WT control sample has not been processed. In other embodiments, the condition only depends on a WT control.

If in block B1040 the one or more genotyping devices determine that, depending on the embodiment, a WT control sample has been processed and the sample to be analyzed is neither a WT control sample nor a NTC sample, then the flow moves to block B1044. In block B1044, preprocessing is performed on the melting curve of the tested sample, whose genotype needs to be determined, and the negative derivative curve of the tested sample is provided (e.g., calculated based on the melting curve). Next, in block B1046, the one or more genotyping devices use the Van't Hoff mixture model to generate a background-corrected negative derivative curve of the sample. The flow then moves to block B1048, where the curve quality of the background-corrected negative derivative curve is calculated.

In block B1050, the one or more genotyping devices determine if the curve quality of the background-corrected negative derivative curve is acceptable. If it is not acceptable, then the flow moves to block B1052, ‘Invalid’ is assigned to the sample, and then the flow proceeds to block B1058, where the flow either stops or returns to block B1002 to continue with the next tested sample whose genotype needs to be determined. If the one or more genotyping devices determine that the curve quality of the background-corrected negative derivative curve is acceptable, then the flow moves to block B1054.

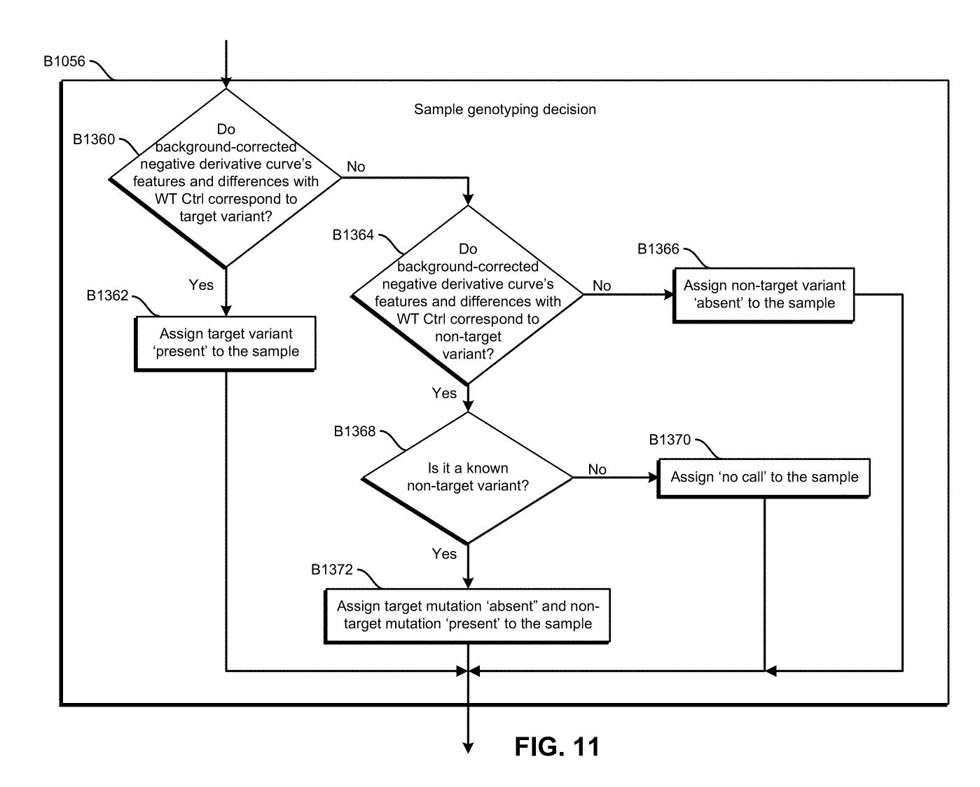

In block B1054, the one or more genotyping devices compare the background-corrected negative derivative curve of the sample to that of the WT control sample. The one or more genotyping devices may compare the features of the background-corrected negative derivative curve of the sample to that of the WT control sample and identify the differences between their features. Next, in block B1056, based on the comparison, the one or more genotyping devices assign a genotype to the sample, for example by applying the criteria in Table 4. An example embodiment of block B1056 is illustrated in FIG. 11. After block B1056, the flow proceeds to block B1058, where the flow either stops (e.g., if all samples have been processed) or returns to block B1002 to continue with the next tested sample's melting curve.

FIG. 11 illustrates an example embodiment of an operational flow for assigning a genotype to a sample. This operational flow can be implemented by one or more genotyping devices. The flow moves to block B1056, which includes blocks B1160-B1172. Then the flow moves to block B1160, where one or more genotyping devices determine if the background-corrected negative derivative curve's features and the differences between the background-corrected negative derivative curve's features and the features of the WT control sample's background-corrected negative derivative curve correspond to the target variant genotype. If yes, then the flow moves to block B1162, where target variant ‘present’ is assigned to the sample, and then the flow exits block B1056. If not, then the flow moves to block B1164.

In block B1164, the one or more genotyping devices determine if the background-corrected negative derivative curve's features and the differences between the background-corrected negative derivative curve's features and the features of the WT control sample's background-corrected negative derivative curve correspond to a non-target variant genotype. If not, then the flow moves to block B1166, where non-target variant ‘absent’ is assigned to the sample, and then the flow exits block B1056. If yes, then the flow moves to block B1168.

In block B1168, the one or more genotyping devices determine if the non-target variant genotype is known. If not, then the flow moves to block B1170, where ‘no call’ is assigned to the sample, and then the flow exits block B1056. If yes, then the flow moves to block B1172. In block B1172, the one or more genotyping devices assign target mutation ‘absent’ and non-target mutation ‘present’ to the sample, and then the flow exits block B1056.

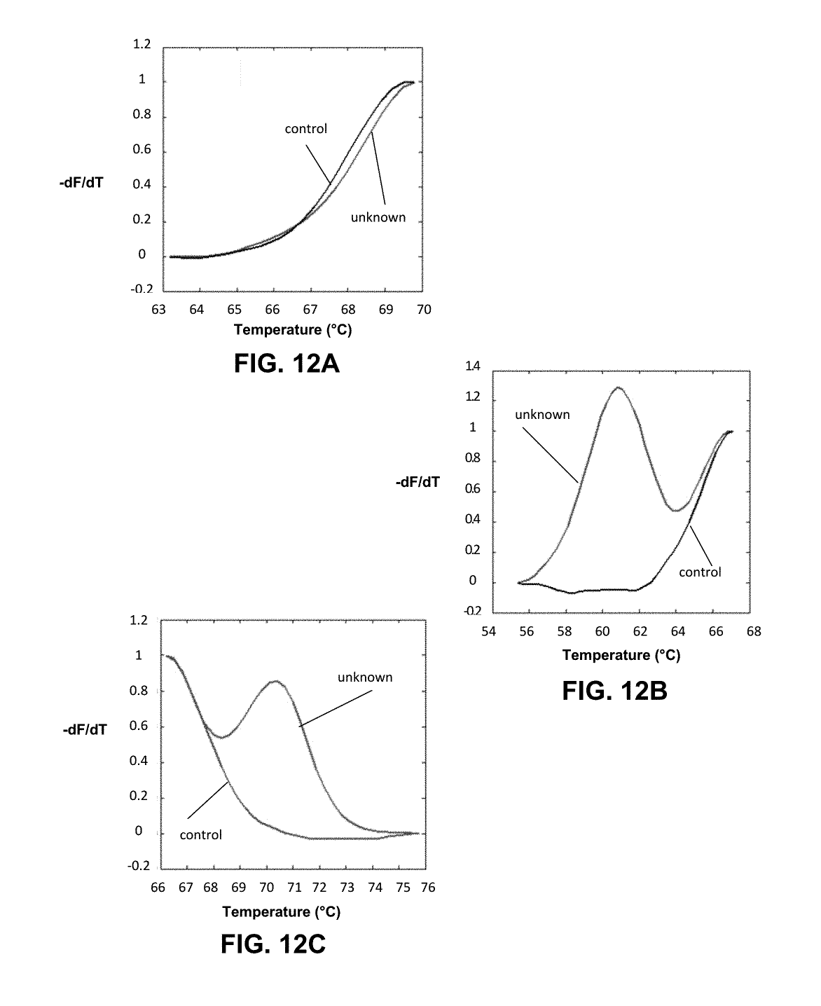

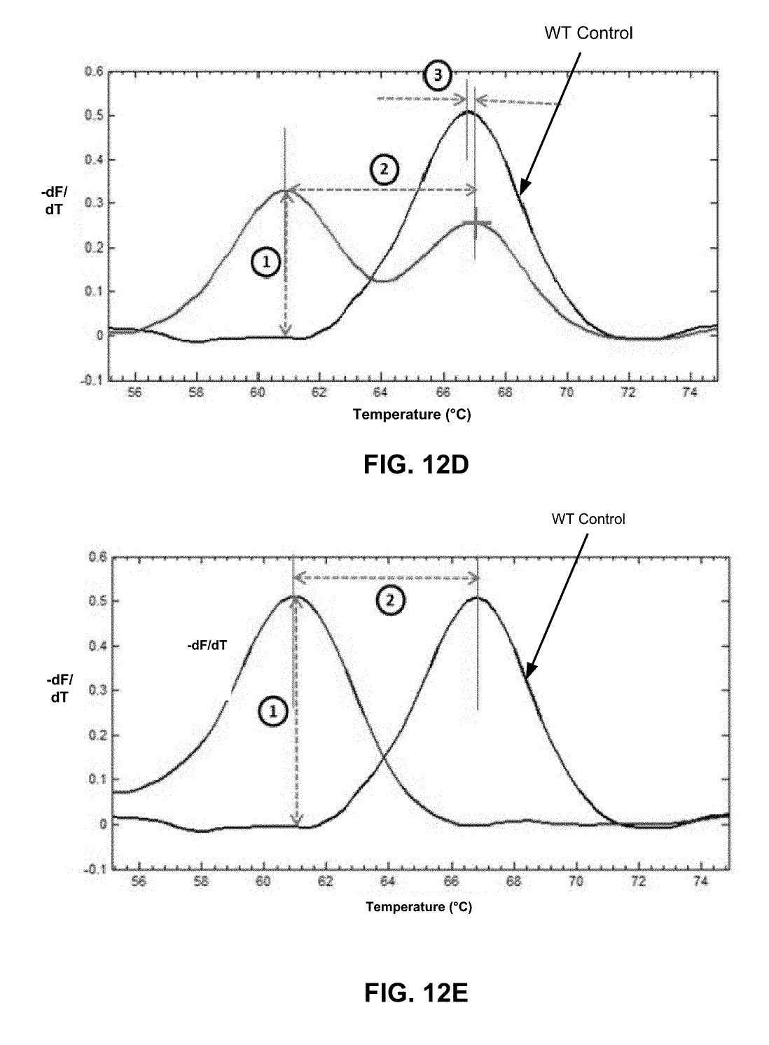

FIG. 12A illustrates an example embodiment of a left-sided negative-derivative-curve comparison between a WT CTRL sample and a tested sample whose genotype needs to be determined. This unknown sample was determined to have only one product reaction through the use of the Van't Hoff mixture model by an embodiment of an automatic genotyping system. In this embodiment, the difference in shape between the unknown sample's background-corrected negative derivative curve and the WT CTRL sample's background-corrected negative derivative curve does not satisfy the HET criteria, thereby eliminating the HET genotype for this unknown sample. In this example, the unknown sample's genotype is HOM.

FIGS. 12B-C illustrate example embodiments of left-sided and right-sided curve comparisons between the background-corrected negative derivative curves of a WT CTRL sample and unknown samples. Each unknown sample was determined to have two product reactions by an embodiment of an automatic genotyping system that used the Van't Hoff mixture model. In these examples, the two unknown samples are different HETs for a probe-based assay. In both cases, a remarkable curve difference in −dF/dT can be observed, and the samples could be classified as a HET depending on the amplitude of their curve differences and the corresponding temperatures where their curve differences occurs.

FIGS. 12D-E illustrate example embodiments of curve features that may be used to determine the genotype of a sample. The assay in this example is probe-based. The genotype is HET in FIG. 12D and is HOM in FIG. 12E. During a primary comparison with the WT CTRL sample during a HET decision (FIG. 12D), the secondary decision to definitely assign or not the sample to a HET genotype is made based on one or more of three curve features: (1) the maximum fluorescence difference between the negative derivative curves or the background-corrected negative derivative curves of the unknown sample and the WT control sample, (2) the difference between the temperature of the major peak of the negative derivative curve or the background-corrected negative derivative curve of the unknown sample (determined as being the closest peak in temperature to the WT control sample) and the temperature of the maximum fluorescence difference between the negative derivative curve or the background-corrected negative derivative curve of the unknown sample and the negative derivative curve or the background-corrected negative derivative curve of the WT control sample, and (3) the temperature peak difference between the negative derivative curve or the background-corrected negative derivative curve of the WT control sample and the major reaction of the negative derivative curve or the background-corrected negative derivative curve of the unknown sample (determined as being the closest peak in temperature to the major peak of the negative derivative curve or the background-corrected negative derivative curve of the WT control sample). If all three curve features are within ranges defined in the configuration information, then the sample is assigned the HET genotype, and a genotype probability of the sample may be derived.

When the primary comparison with the WT CTRL eliminates a HET assignment, or if the three curve features do not meet the ranges defined in the configuration information, then either a HOM or WT genotype is considered as genotype for the sample. In this situation, two curve features are used: (1) the maximum fluorescence amplitude of the negative derivative curve or the background-corrected negative derivative curve of the unknown sample and (2) the temperature difference between the peak of the negative derivative curve or the background-corrected negative derivative curve of the WT CTRL sample and that of the negative derivative curve or the background-corrected negative derivative curve of the unknown sample. These curve features are then compared to the criteria that are defined in the configuration file in order to decide whether the sample is either a WT or a HOM genotype (FIG. 12E). If none of the above criteria are met, then the sample may be assigned to a no-call or NA (e.g., undetermined genotype, or undefined genotype not included in the configuration file).

In some embodiments, the automated genotyping operations rely on a set of pre-defined parameters that are contained in configuration information. These pre-defined parameters are assay-dependent, which means that, for any new assay, configuration information may need to be generated. These parameters may be derived theoretically and knowing a priori the variance of the acquisition device for sample melts, or using a training set of samples whose genotypes are known. When using a training set of samples, the parameters can be derived using basic statistical measures over the training set of relevant sample features, such as the mean value X̅ and standard deviation σ, and where X̅ may be, for example, the difference in melting temperature between the genotype-known HOM samples and WT CTRL samples. These statistical measures are included in the configuration information for the considered assay and allow setting each genotype's boundaries, for example X̅±3σ. Thereby, when the automated genotyping operations are used in a non-training mode and applied to a tested unknown sample, the tested unknown sample will typically be assigned a genotype if its negative derivative curve's features are within the boundaries of that genotype.

While using a training set of samples for generating the configuration information for a new assay, it may be assumed that the assay was designed chemistry-wise in a manner such that no overlap between known genotype boundaries occurs. However, such an assumption may not always be valid, and some overlap between genotype boundaries (e.g., overlap with the reference WT genotype) may exist. In this situation, the mean of the non-reference (non-WT) genotype may be shifted so that no overlap between genotype boundaries occurs.

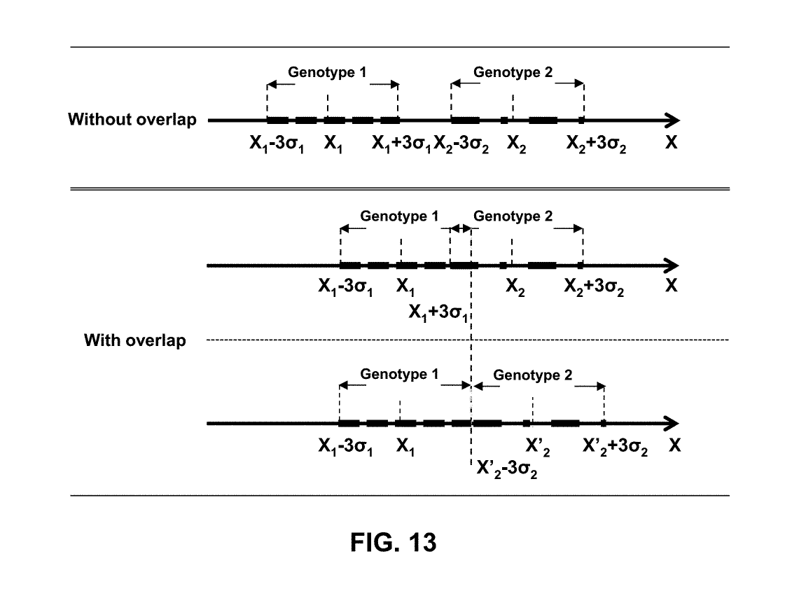

FIG. 13 illustrates example embodiments of statistical measures that may be used as criteria for genotyping a sample. The statistical measures may define genotype boundaries that can be used whether there is overlap between the genotypes. In this example embodiment the boundaries of genotypes are set to X̅±3σ. For example, in FIG. 13, in the “Without overlap” example, the mean X̅ of genotype 1 is X₁, and the boundaries of genotype 1 are X₁±3σ₁. Also, the mean X̅ of genotype 2 is X₂, and the boundaries of genotype 2 are X₂±3σ₂. The boundaries of genotype 1 and genotype 2 do not overlap, so no correction is needed and the statistical measures are stored as-is in the configuration information.

Furthermore, in FIG. 13, in the “With overlap” example, the boundaries of genotype 1 and genotype 2 do overlap. Because genotype 1 is the reference genotype, the boundaries of genotype 2 are shifted so that genotype 1 and genotype 2 do not overlap. Thus, the mean X̅ of genotype 2 becomes X′₂ and the boundaries of genotype 2 become X^{′}₂±3σ₂. These corrected values may be stored in the configuration information.

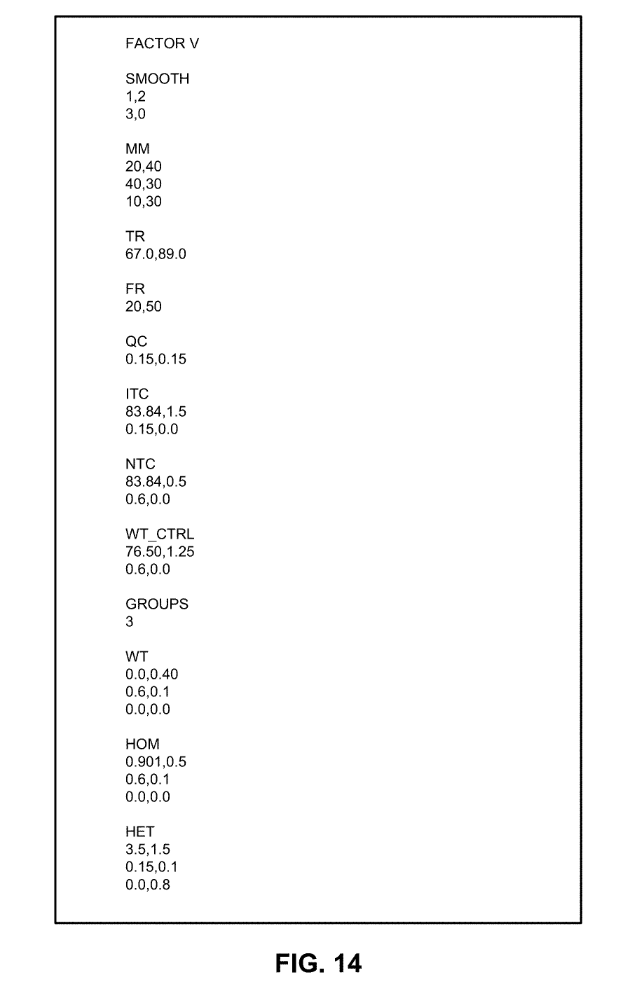

FIG. 14 illustrates an example embodiment of a configuration file. The definitions of each field for WT samples and HOM samples are as follows:

| WT (or HOM) | Genotype Name |
|---|---|
| 0.0^{(a)}, 0.40^{(b)} | (a) Average difference between (WT Tₘ − ITC Tₘ) and |
| | (WT CTRL Tₘ − ITC Tₘ). If ITC is not present, then |
| | ITC Tₘ is set to 0; and |
| | (b) 3x standard deviation of the difference. |
| 0.6^{(c)}, 0.1^{(d)} | (c) Average model amplitude at Tₘ; and |
| | (d) 3x standard deviation of the model amplitude. |
| 0.0^{(e)}, 0.0^{(f)} | (e) Not used/Not applicable; and |
| | (f) Not used/Not applicable. |

For HET samples, each field is defined as following:

| HET | Genotype Name |
|---|---|
| 3.5^{(a)}, 1.50^{(b)} | (a) Average difference between the major peak Tₘ and the |
| | minor peak Tₘ; and |
| | (b) 3x standard deviation of the difference. |
| 0.15^{(c)}, 0.1^{(d)} | (c) Average model amplitude at the minor peak Tₘ; and |
| | (d) 3x standard deviation of the model amplitude. |
| 0.0^{(e)}, 0.8^{(f)} | (e) Average difference between major sample peak Tₘ and |
| | WT CTRL Tₘ; and |
| | (f) 3x standard deviation of the difference. |

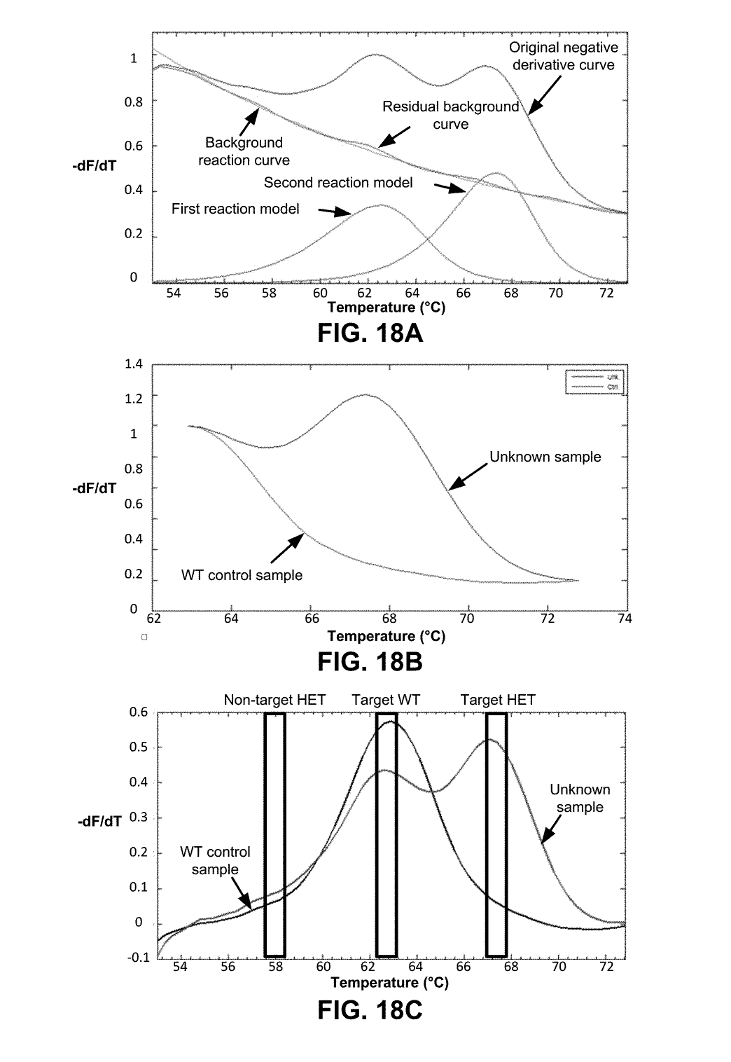

In the embodiment in FIG. 18, the configuration file includes a third field of parameters that allows recognition of different HET-genotype characteristics when the assay is designed to detect more than one variant along the DNA sequence of interest. The parameters in this third field represent the average difference and the range where the major peak of the HET-genotype sample is expected with respect to that of the WT CTRL sample. This third field is not applicable for WT and HOM genotypes and is set to 0.0.

Also some embodiments of a configuration file include other information. For example, some embodiment include a fourth field that describes averaged thermodynamic parameters (e.g., a total enthalpy change ΔH, a melting temperature) of each genotype. They can also be used to optimize sample processing and thereby obtain results from the Van't Hoff mixture model more quickly. Some embodiments use a Van't Hoff mixture model (MM) fitting to determine the underlying reaction models of the DNA sample. The Van't Hoff MM fitting is based on the Van't Hoff equation, which can approximately relate the equilibrium constant K of a DNA sample that is denatured (from double strand to single strand) according to the following equation of free energy ΔG:

ΔG=−RT ln K, (1)

where R is the ideal gas law constant, and where T is the measured temperature in Kelvin.

Also, from the definition of Gibbs free energy,

ΔG=ΔH−TΔS, (2)

where ΔH is the total enthalpy change and ΔS is the entropy.

These equations lead to an equation that describes the equilibrium constant K as a function of the measured temperature T, or K(T):

$$K ( T ) = exp ( \frac{Δ S}{R} - \frac{Δ H}{RT} ) . \qquad ( 3 )$$

The equilibrium constant K can be defined by the concentrations of double-stranded DNA and single-stranded DNA, where double-stranded DNA is denoted as AA′, and where single-stranded DNA is denoted as A and A′ for the forward and reverse strands, respectively. Thus, for the reaction AA′ [symbol] A+A′, the equilibrium constant K may be described in terms of the concentrations (denoted by square brackets) according to

$$K ( T ) = \frac{{[ {AA}^{′} ]}_{T}}{{{[ A ]}_{T} [ {A}^{′} ]}_{T}} . \qquad ( 4 )$$

[X]_{T} is adopted to signify the concentration of X (in equation (4), X is A, A′, and AA′) at temperature T.

However, the total concentration does not change with temperature. Thus the total concentration can be used as the initial double-stranded DNA concentration at low temperatures. This is described by the following:

$${C}_{TOT} = {[ {AA}^{′} ]}_{T} + \frac{{[ A ]}_{T} + {[ {A}^{′} ]}_{T}}{2} = {[ {AA}^{′} ]}_{T} + {[ A ]}_{T} . \qquad ( 5 )$$

Note that the single-stranded concentrations of the forward and reverse strands are equal: [A]_{T}=[A′]_{T}.

At each temperature, the normalized fluorescence of the DNA is the concentration of double-stranded DNA normalized by the initial low-temperature double-stranded DNA concentration. The fluorescence signal F(T) can be described by the following:

$$F ( T ) = \frac{{[ {AA}^{′} ]}_{T}}{{[ {AA}^{′} ]}_{T} + {[ A ]}_{T}} . \qquad ( 6 )$$

Therefore, C_{TOT}F(T)=[AA′]_{T} and C_{TOT}[1−F(T)]=[A]_{T}, and K(T) can be described in terms of F(T) and C_{TOT}:

$$K ( T ) = \frac{{[ 1 - F ( T ) ]}^{2} {C}_{TOT}}{F ( T )} . \qquad ( 7 )$$

Using the dissociation temperature Tₘ of the DNA, which is a critical temperature point of the DNA melt and is defined as the temperature such that half of the DNA has been denatured, or in other words F(Tₘ)=1/2, equation (7) simplifies to K(Tₘ)=C_{TOT}/2.

Using the difference of the Van't Hoff equation (1) at two separate temperature instances T₁ and T₂, then

$$ln [ \frac{K ( {T}_{2} )}{K ( {T}_{1} )} ] = \frac{Δ H}{R} ( \frac{1}{{T}_{1}} - \frac{1}{{T}_{2}} ) . \qquad ( 8 )$$

And using equations (7) and (8) with the melting temperature Tₘ for T₁ and with the measured temperature T for T₂ produces the following:

$$\frac{{2 [ 1 - F ( T ) ]}^{2}}{F ( T )} = exp [ \frac{Δ H}{R} ( \frac{1}{{T}_{m}} - \frac{1}{T} ) ] . \qquad ( 9 )$$

The previous expression can be defined as the equilibrium constant to melt equilibrium constant ratio h(T):

$$h ( T ) = exp [ \frac{Δ H}{R} ( \frac{1}{{T}_{m}} - \frac{1}{T} ) ] . \qquad ( 10 )$$

Also, expanding equation (9) produces the following binomial equation of the fluorescence signal F(T):

2F²(T)−(4+h(T))F(T)+2=0. (11)

And equation (11) has the following solutions:

$$F ( T ) = \frac{4 + h ( T ) \pm \sqrt{{h}^{2} ( T ) + 8 h ( T )}}{4} . \qquad ( 12 )$$

Because h(Tₘ)=1, only one solution (the smaller solution) generates the desired value of F(Tₘ)=1/2. Thus,

$$F ( T ) = \frac{4 + h ( T ) - \sqrt{{h}^{2} ( T ) + 8 h ( T )}}{4} . \qquad ( 13 )$$

Equation (13) provides a function for modeling melt fluorescence (a melt fluorescence model for a product reaction AA′ [symbol] A+A′) that depends on just 2 parameters: the total enthalpy change ΔH and the melting temperature Tₘ. Furthermore, the second parameter is easily interpretable, and the first parameter can be predicted based on experimentally-obtained parameters of DNA melting models.

Also, the fluorescence signal has the following limits:

$${lim}_{T \to {0}^{+}} F ( T ) = 1. \qquad ( 14 )$$

This can be seen because the limit of h(T) as T→0⁺ is zero. Low temperatures should produce 100% double-stranded DNA and maximum fluorescence. Also note that

$${lim}_{T \to ∞} F ( T ) \approx 0. \qquad ( 15 )$$

While the ideal function would go to zero at very high temperatures, the fluorescence model doesn't go quite to zero. Before considering the convergence of the fluorescence signal F(T), first consider h(T), which converges to a non-zero value h(∞):

$$h ( ∞ ) = {lim}_{T \to ∞} h ( T ) = exp ( \frac{Δ H}{R} \frac{1}{{T}_{m}} ) . \qquad ( 16 )$$

For two base pairs, typically the total enthalpy change ΔH is approximately 35,000 J/mol, the ideal gas law constant R is approximately 8.3 J/mol K, and the melting temperature Tₘ is approximately 350 K. This gives h(∞)≈exp(12)≈162,000. Inserting this value into equation (13) produces, for this rough example, F(∞)≈0.00001. In longer DNA sequences the total enthalpy change ΔH will increase, making the fluorescence signal F(T) exponentially smaller.

From the fluorescence signal F(T), an approximate DNA fluorescence probability density with respect to temperature can be generated. This probability density represents the distribution p(T) over temperature for a DNA melt (disassociation or association) event. In some embodiments, the density p(T) is the derivative (e.g., a negative derivative) of 1−F(T). This is the negative derivative of the fluorescence signal F(T), which can be described as follows:

$$p ( T ) = \frac{d}{dT} [ 1 - F ( T ) ] = F ( T ) \frac{Δ H}{R} \frac{1}{{T}^{2}} \frac{h ( T )}{\sqrt{{h}^{2} ( T ) + 8 h ( T )}} , \\ p ( T ) = \frac{Δ H}{R} \frac{h ( T )}{4 {T}^{2}} [ \frac{4 + h ( T )}{\sqrt{{h}^{2} ( T ) + 8 h ( T )}} - 1 ] . \qquad ( 17 )$$

This provides a theoretical functional model for the melt profile of homogeneous samples of DNA. For heterogeneous samples (e.g., heterozygous DNA), the melt profile would be a mixture of two such functions with different parameters.

However, some properties (like the mean and the variance) of the negative derivative of the fluorescence p(T) (from the above formulation) may be computationally expensive, as indicated by the cumbersome nature of equation (17). But the median temperature is the melting temperature Tₘ because the cumulative distribution is 1/2 at the melting temperature Tₘ. Furthermore, the equations may be slightly more amenable to analysis if the domain is inverse temperature instead of temperature.

Also, one important characteristic of the negative derivative of the melt fluorescence signal F(T) is the location of the peaks. This is the mode of the melt. This can be obtained by differentiating the negative derivative of the fluorescence p(T) with respect to the measured temperature T or 1/T, setting the derivative equal to zero, and solving the equation for the measured temperature T. In some embodiments, the peak of the distribution occurs at peak temperature Tₚₖ:

$${T}_{pk} = {T}_{m} \frac{1}{1 + {T}_{m} \frac{R}{Δ H} ln ( \frac{1 + \sqrt{2}}{4} )} \approx {T}_{m} \frac{1}{1 - \frac{{T}_{m}}{2} \frac{R}{Δ H}} . \qquad ( 18 )$$

Thus, the peak temperature Tₚₖ, which is the temperature at the peak of the negative derivative of the fluorescence curve, is slightly higher than the melting temperature Tₘ. In preliminary experiments that used embodiments of an ITC (internal temperature control with a known melting temperature) DNA sequence, a peak temperature of about ½ degree higher than the melting temperature was observed.

Some devices, systems, and methods use a mixture model to model the raw fluorescence curve. Also, some embodiments of the mixture model assume that there are M or fewer independent reactions that influence the fluorescence, and the total observed fluorescence is a mixture of these individual effects. Some embodiments of the mixture model can be described mathematically as follows:

$${F}_{total} ( T ; Θ ) = {\sum }_{i = 1}^{M} {α}_{i} {F}_{i} ( T ; {Θ}_{i} ) such that {\sum }_{i = 1}^{M} {α}_{i} = 1 and {α}_{i} \geq 0 for all i , \qquad ( 19 )$$

where F^{total}(T) is the total fluorescence (and should match the observed data if the model is good), where Fᵢ(T; Θᵢ) is the fluorescence of the i^{th} reaction as a function of temperature, where Θᵢ is the set of parameters for the i^{th} fluorescence model, where the mixture coefficient αᵢ is the contribution of Fᵢ(T; Θᵢ) (mixture coefficient αᵢ is also referred to as “contribution Δᵢ,” and Fᵢ(T; Θᵢ) is also referred to as “model i”) to the total model (mixture coefficient Δᵢ is the weight factor of model i to the total reaction), and where Θ is the collection of all parameters {αᵢ, Θᵢ:i∈1, . . . , M}. Furthermore, the constraints indicate that each model has some non-negative contribution to the total and that individual model contributions sum to 1. And a mixture model that is based on the Van't Hoff equation (the Van't Hoff equation forms the basis of Fᵢ(T; Θᵢ), which is the fluorescence profile of independent reaction i to the overall fluorescence) is referred to herein as the Van't Hoff mixture model.

The previous description presents a melt model that had two parameters: the melting temperature Tₘ and the total enthalpy change ΔH of the reaction. Thus, for M reactions, some embodiments have 3M−1 parameters, including the M−1 choices for the contribution αᵢ values (note that the constraint fixes one contribution αᵢ value given the other values).

Additionally, if the background fluorescence is also a reversible reaction, and if the ITC is a reversible reaction, then a homozygous (wild-type and variant) genotype will require M=3, and a heterozygous genotype will require M=4 (or more). Thus, for 4 reactions the model requires the determination of 11 parameters (2 for each reaction model and a mixture coefficient for each reaction model, where the last reaction mixture coefficient can be determined from the others because they all sum to 1).

Furthermore, consider some other common reactions that possibly affect the fluorescence. For example, the unbound fluorescence dye itself may be involved in a reversible reaction whereby the level of fluorescence changes before and after the reaction. Additionally, some parts of the solution may be relatively inert, so their fluorescence is unaffected by temperature. Other reactions may be irreversible. Below is a summary of some possible reaction models:

TABLE 5

| Reaction | Example | Fluorescence Model F(T) | Parameters |
|---|---|---|---|
| DNA double- stranded to single- stranded type-2 | AA′ [symbol] A + A′ | $F ( T ) = \frac{4 + h ( T ) - \sqrt{{h}^{2} ( T ) + 8 h ( T )}}{4}$ $h ( T ) = exp [ \frac{ΔH}{R} ( \frac{1}{{T}_{m}} - \frac{1}{T} ) ]$ | ΔH and Tₘ |
| Single agent change type-1 | B [symbol] C | $F ( T ) = \frac{1}{1 + h ( T )}$ $h ( T ) = exp [ \frac{ΔH}{R} ( \frac{1}{{T}_{m}} - \frac{1}{T} ) ]$ | ΔH and Tₘ |
| No reaction (inert) | D [symbol] D | F(T) = 1 | None |
| type-0 | | | |
| Irreversible | E → F | F(T) = e^{-T}^{²}^{/2(T}^{ₘ}⁾^{²} | Tₘ |
| reaction | | | |
| type NR | | | |

These models are also applicable to the negative derivative, as all of these individual models are differentiable. However, the inert components do not contribute to the negative derivative because the derivative of the constant fluorescence signal F(T)=1 is zero.

Several techniques to estimate the parameters of the model exist. For example, one of these techniques is Expectation Maximization (EM). Expectation Maximization is a technique for solving the parameters of a mixture model. In this technique, two alternating steps are performed on the model until convergence (or until a certain number of steps have been performed). The standard form uses observed samples that are assumed to be drawn from some unknown mixture distribution. First, initial guesses of the parameters of this distribution are made, and then the following two steps are repeated:
1. Expectation step: calculates the probability of the observation given that it was drawn from each of the individual distributions that make up the mixture and given the distribution and mixture parameter estimates.
2. Maximization step: finds the maximum likelihood estimates of the distribution parameters given the set of observations where the contribution of each sample to each sub-model (e.g., each independent reaction) is based on the probability that the observation originated from that model (as estimated in the Expectation step).
   The technique is defined for samples that are drawn from a distribution.

However, this technique essentially measures the distribution itself from the negative derivative of the fluorescence. Thus, some embodiments treat the EM problem like having a relative number of reaction “samples” at each temperature. The relative number of “samples” is proportional to the negative derivative of the fluorescence. One caution in this technique is that, because the pseudo-samples are coming from a range of temperatures, some embodiments need to modify the underlying theoretical distribution to account for the fact that they are “drawing” samples from a truncated Van't Hoff distribution, not from a complete Van't Hoff distribution (here the melt-temperature probability is referred to as the Van't Hoff distribution; for example, for a type-2 reaction, the distribution takes the form of equation (17); while this function is only approximately a true distribution, it can be treated as a probability distribution, and when examined in a truncated form, it becomes a valid distribution).

However, some embodiments of EM have limitations. First, because it is a descent-type technique, it can easily converge to a solution that is a local minimum instead of a global minimum. Therefore, EM can be sensitive to the choice of the initial parameters. If these initial parameters are chosen poorly, the global optima may be unreachable. Examples of operations for choosing the initial parameters include the following:
1. For applications relating to automated genotyping, some embodiments will know a priori the approximate parameters that the embodiments will estimate, given the genotype of the PCR-generated genetic material. Thus, these embodiments can start their search around the expected parameters.
2. Some embodiments add additional mixture components so that they may more generally fit a broader range of melting curves. This runs the risk of over-fitting the model to the data. However, this risk can be mitigated through the use of regularization terms on the mixture-coefficient estimation or through a reaction-pruning process that tests the effects of eliminating reactions from the mixture model.
3. Some embodiments use a non-EM algorithm or a stochastic EM-type algorithm to estimate parameters. For example, some embodiments use Markov Chain Monte Carlo to estimate parameters.

Second, the maximization step requires some embodiments to know, or be able to reasonably derive, maximum-likelihood estimators. In some embodiments, such as embodiments that use Gaussian mixture models, maximum-likelihood (ML) estimates are easily obtained. However, in some embodiments, the distributions are not in a form that is conducive to ML estimation, at least in closed form. Some embodiments effectively overcome this problem by using optimization packages and using numerical derivatives.

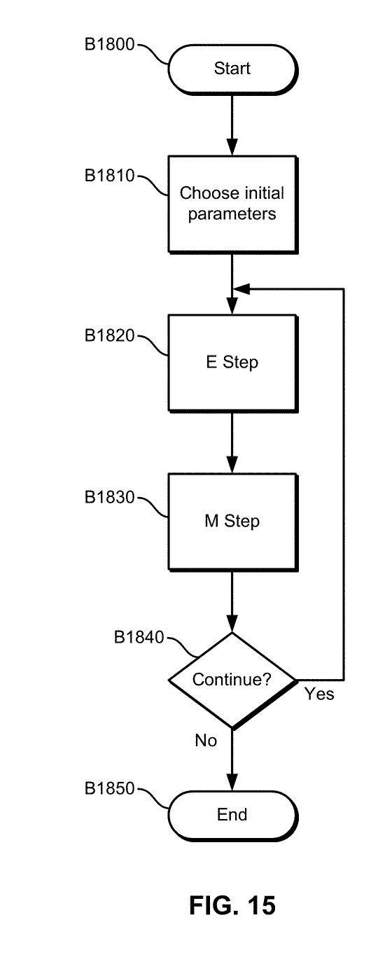

FIG. 15 illustrates an example embodiment of an operational flow for Expectation Maximization. The operational flow may be implemented by a specially-configured system or device (e.g., an automatic genotyping system). After starting in operation B1500, the automatic genotyping system chooses the initial parameters in operation B1510. After the initial parameters have been determined, the automatic genotyping system next executes the expectation step (E-step) in operation B1520, then the maximization step (M-step) in operation B1530. Then in operation B1540, the automatic genotyping system repeats operation B1520 and operation B1530 for a certain number of iterations or until the algorithm converges, and then the flow ends in block B1550.

In some embodiments, during the expectation step (E-step) in block B1520, the automatic genotyping system calculates the data memberships to each of the mixture-basis classes. That is, at any given temperature, the goal is to calculate how many of the occurring reactions are attributable to each of the underlying independent reactions. The membership to the reaction class k (mixture-basis class k) at a temperature t can be described by the following:

$${w}_{t , k} = \frac{{α}_{k} {p}_{k} ( t \vert {Θ}_{k} )}{{\sum }_{i = 1}^{M} {α}_{i} {p}_{i} ( t \vert {Θ}_{i} )} , \qquad ( 20 )$$

where pₖ(t|Θₖ) is the truncated Van't Hoff distribution, which comes from equation (17) but is renormalized so that the function integrates to 1 in the temperature ranges being fit by the model. Also,

$${p}_{k} ( t \vert {Θ}_{k} ) = \frac{{p}_{VH} ( t \vert {Θ}_{k} )}{{\int }_{{T}_{L}}^{{T}_{H}} {p}_{VH} ( t \vert {Θ}_{k} ) dt } , and \\ {p}_{k} ( t \vert {Θ}_{k} ) = \frac{{p}_{VH} ( t \vert {Θ}_{k} )}{{F}_{VH} ( {T}_{H} \vert {Θ}_{k} ) - {F}_{VH} ( {T}_{L} \vert {Θ}_{k} )} , \qquad ( 21 )$$

where F_{VH}(t|Θₖ) is the cumulative distribution of the non-truncated Van't Hoff distribution.

The parameters of the distribution Θₖ include the reaction type and the parameters in the last column of Table 5. In some embodiments of the mixture model, the reaction type is assumed to be fixed, but the parameters and the mixture coefficients αᵢ are estimated.

In the maximization step (M-step) in block B1530, the mixture coefficients are calculated (e.g., estimated) to obtain the maximum-likelihood estimates of the reaction functions. There are a few technical challenges that are addressed to accomplish these operations. These challenges are described below.

First, to estimate the mixture coefficients, some embodiments perform a constrained optimization to solve the constrained least squares problem:

$${min}_{x} {‖ Ax - b ‖}^{2} subject to {x}_{i} \geq 0 for all i ε \{ 1 , … , M \} and \\ {\sum }_{i = 1}^{M} {x}_{i} = 1. \qquad ( 22 )$$

This unmixing problem can be solved using the Lagrange multiplier theory.

The second challenge is overcoming the generation of the maximum-likelihood (ML) estimates of the distribution parameters for each distribution. Typically, ML estimation is based on a set of samples drawn from the distribution of interest. However, here the melting process generates the fluorescence curve, which essentially measures one minus the cumulative distribution. So to perform ML estimation, some embodiments assume that the number of samples drawn at each sample temperature is proportional to the negative derivative of the fluorescence. With a set of temperatures and negative derivative fluorescence observations Z={(tⱼ,fⱼ):j∈{1, . . . , N}}, some embodiments operate as though there are C×fⱼ×wₜ_{ⱼ}_{,k} samples at each temperature tⱼ (where C is a constant). And furthermore, these samples can be assumed to be drawn from the truncated model distribution. Thus, the probability of all of the samples can be described according to

$$p ( Z \vert {Θ}_{k} ) = {\prod }_{j = 1}^{N} {[ p ( {t}_{j} \vert {Θ}_{k} ) ]}^{{Cf}_{j} {w}_{{t}_{j} , k}} . \qquad ( 23 )$$

If this is converted to the log likelihood and maximized, it produces the following:

$${max}_{{Θ}_{k}} log p ( Z \vert {Θ}_{k} ) = {max}_{{Θ}_{k}} {\sum }_{j = 1}^{N} {Cf}_{j} {w}_{{t}_{j} , k} log p ( {t}_{j} \vert {Θ}_{k} ) . \qquad ( 24 )$$

Note that this is equivalent to

$${min}_{{Θ}_{k}} {\sum }_{j = 1}^{N} {f}_{j} {w}_{{t}_{j} , k } log \frac{{f}_{j} {w}_{{t}_{j} , k }}{p ( {t}_{j} \vert {Θ}_{k} )} , \qquad ( 25 )$$

and this expression is the Kullback-Leibler divergence: D_{KL}(f·w∥p). This is a measure of how well p fits the distribution given by f·w, or more precisely, the measure of information loss when the theoretical distribution is used to approximate the observed data.

The optimization problem in equation (24) can be solved using gradient-descent function minimization, which minimizes a continuously-differentiable function. One issue with gradient-descent function minimization is the need for the partial derivatives of the truncated Van't Hoff distribution with respect to the parameters. While the derivatives can be obtained, they are quite long and contain many terms in their expressions. Thus, some embodiments use numerical derivatives at a particular location by evaluating the distribution at a particular parameter setting and then at the same parameter setting plus some small epsilon. Some embodiments use an epsilon of 10e−6 for both melting temperature Tₘ and total enthalpy change ΔH parameters, and then divide the difference of these two values by epsilon to estimate the derivative.

Additionally, some embodiments run the optimization for a predefined number of iterations or until convergence, but often the algorithm is mostly converged after just a few iterations. So to save time, some embodiments limit the number of iterations to 10. This probably does not cause a problem because this EM process is repeated several times until convergence.

Furthermore, following is an example of a technique to select the starting parameters of the mixtures and the underlying reaction models. The technique cross-correlates the fluorescence negative-derivative data to a reaction model curve (type-2) with a high total enthalpy change ΔH and a typical melting temperature Tₘ. Some embodiments use the melting temperature Tₘ of 350 K and an enthalpy change ΔH of 6000 kJ/mol. This essentially provides a narrow temperature-reaction curve that acts as a smoothing filter on the original negative-derivative data. The rationale for this technique is to treat this prototypical reaction as a matched filter that can be used for detection. This narrow reaction curve helps to avoid over-smoothing the data so that no substantial loss of information (e.g., shape-wise) occurs from the smoothing.

The smoothing kernel is shifted by the melting temperature Tₘ so that it is centered at 0, and cyclic cross-correlating is performed. This is effectively carried out by multiplying the fast Fourier transform (FFT) of the negative derivative and the FFT of the smoothing kernel. The inverse FFT of the product produces the cross correlation of the two curves.

In order to perform this operation, it may be necessary to re-sample the negative-derivative curve to uniform temperature samples. To resample, some embodiments use simple linear interpolation at the desired temperature points, or some other interpolation methods, like polynomial fitting, which can be done with a Savitzky-Golay (SG) filter. In some embodiments, the re-sampled data comes from the negative derivative of a polynomial fit of the raw fluorescence data (e.g., the SG generated derivative).

From the cross correlation of the smoothing kernel with the negative derivative, an approximate second derivative can be generated. The second difference of the cross-correlation data can be used as the approximate second derivative. The negative second derivative is a measure of concavity. Thus, some embodiments look for parts of the cross correlation that exhibit strong concavity. To determine the strongly concave regions, these embodiments may first estimate the standard deviation of the concavity measure. Assuming that the concavity is more or less random and is not related to some reaction signal, then not many outliers would appear in the distribution of concavity measurements. Positive outliers are of interest because they represent strong changes in the shapes of the reaction curves, which look like peaks.

The reason for looking at concavity instead of just the peaks of the cross-correlation is that in the underlying mixture of reactions, there can be an overlap of the underlying reaction curves, and peaks from reactions don't always manifest themselves as peaks in the cross-correlation because they can be obscured by larger neighboring (in temperature) reactions. The concavity measure may perform better in these cases because a strong concavity signal can still detect these hidden peaks because of the rate of change in the slopes of the curves around the peak.

Note that if the presence of outliers is assumed, then a standard estimate of the random background variations will be influenced by the outliers. Thus, some embodiments use median absolute difference (MAD) to estimate the standard deviation. Also, note that for a normal distribution the standard deviation σ is approximately σ=1.48 median {|zᵢ|:i∈1, . . . N}. Because this measurement uses the median operation, the results are not biased by a few outliers. Some embodiments then search for peaks in the concavity measure that are 3σ above the mean concavity. Because the signal is generated from a cyclic cross-correlation using FFTs, some embodiments throw away any concave outliers that are in the boundary regions of the melt domain—they don't use low or high temperature detections because they are distorted by the kernel cyclic wrapping.

The strengths of the peaks in the concavity measure are used as relative mixture amounts in the starting mixture coefficients. The locations of the peaks are the starting melting temperatures Tₘ used by the EM algorithm. And the initial total enthalpy change ΔH is set to the kernel total enthalpy change ΔH of 6000 kJ/mol.

In addition to these starting components, some embodiments add a background reaction component that has the starting mixture coefficient of 1, a melting temperature Tₘ of 200 K, and a total enthalpy change ΔH of 50 kJ/mol. These parameters are used because the starting background reaction curve is very similar to one minus the logistic function (in inverse temperature). By choosing a low melting temperature Tₘ, these embodiments can examine the tail of the logistic-like function, which looks similar to an inverse exponential function in temperature. The low enthalpy change relates to a slow decay of the function relative to the fluorescence decay of the DNA reactions. These initial values are typically modified by the EM algorithm. In some experiments, the background parameters tend to converge to roughly the same values for a given microfluidic device.

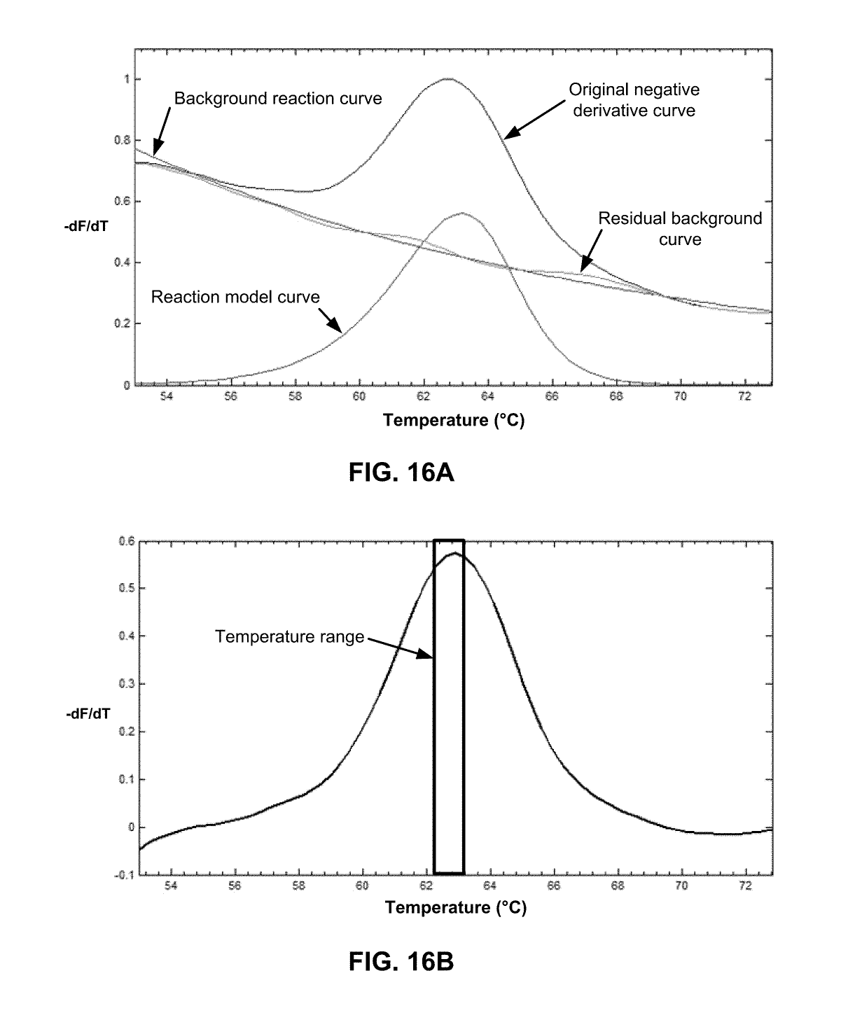

FIG. 16A illustrates an example embodiment of an original negative derivative curve, a background reaction curve, a residual background curve, and a reaction model curve. Once an automatic genotyping system obtains the original negative derivative curve of a DNA sample, the automatic genotyping system can perform mixture-model fitting to identify the background reaction curve and the residual background curve. The automatic genotyping system can then remove the background reaction curve from the original negative derivative curve to generate a background-corrected negative derivative curve that describes the melting of the DNA sample without any background components (e.g., primers, dye).

FIG. 16B illustrates an example embodiment of a temperature range. The temperature boundaries identify a range in which the peak of a WT CTRL negative derivative curve is expected to be found based on parameters in the corresponding assay-configuration information. This can be verified when evaluating a WT CTRL sample's negative derivative curve. In this example, the WT CTRL sample is found valid because the peak of the negative derivative curve of the WT control sample is within the temperature boundaries of a typical WT control sample for the considered assay, as contained in the configuration information.

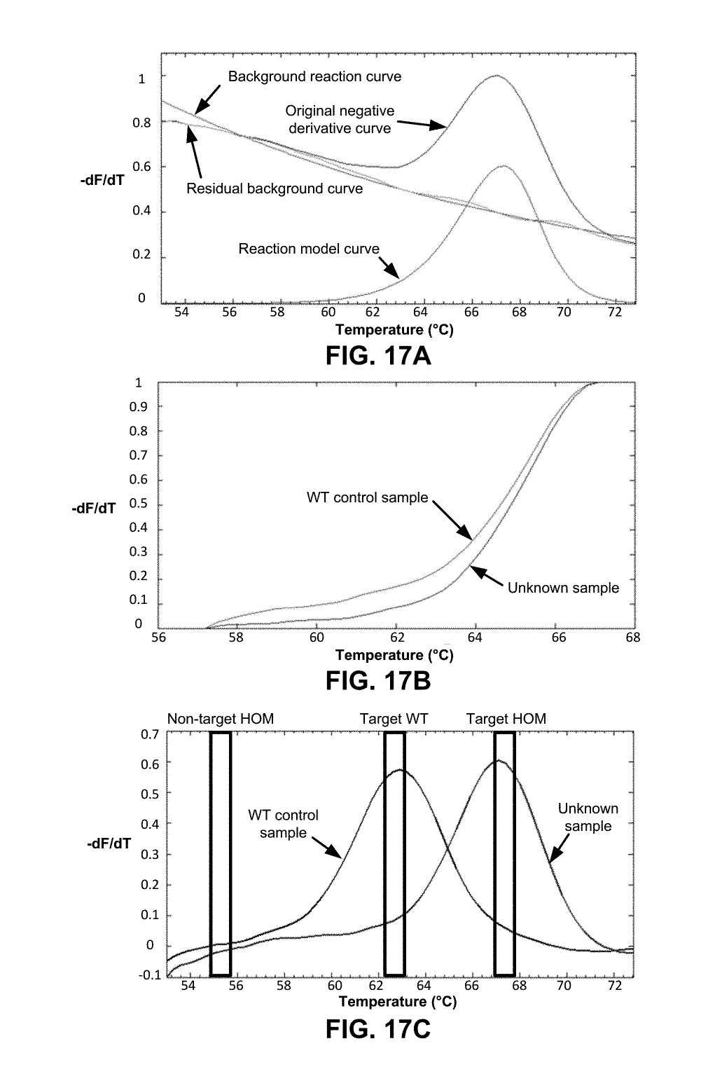

FIG. 17A illustrates an example embodiment of an original negative derivative curve, a background reaction curve, a residual background curve, and a reaction model curve. Once an automatic genotyping system obtains the original negative derivative curve (e.g., by calculating the negative derivative of a melting curve), the automatic genotyping system can perform mixture-model fitting to identify the background reaction curve and the residual background curve. The automatic genotyping system can then remove the background reaction curve from the original negative derivative curve to generate a background-corrected negative derivative curve that describes the disassociation of the DNA sample without any background components (e.g., primers, dye).

FIG. 17B illustrates an example embodiment of a comparison between background-corrected negative derivative curves of a wild-type control sample and a tested unknown sample.

FIG. 17C illustrates an example embodiment of temperature boundaries as a basis for a genotyping decision. This example uses the WT control sample's negative derivative curve from FIG. 16B as the WT control sample's negative derivative curve and uses the reaction model curve from FIG. 17A as the tested unknown sample's reaction model curve. This example also includes three mutations' boundaries, one for a non-target HOM, one for a target WT, and one for a target HOM. An automatic genotyping system may provide a genotyping decision based on the overlap of the temperature difference point within any of the defined mutations' temperature boundaries.

FIG. 18A illustrates an example embodiment of an original negative derivative curve, a background reaction curve, a residual background curve, a first reaction model curve, and a second reaction model curve. Once an automatic genotyping system obtains the original negative derivative curve, the automatic genotyping system can perform mixture-model fitting to identify the background reaction curve and the residual background curve. The automatic genotyping system can then remove the background reaction curve from the original negative derivative curve to generate a background-corrected negative derivative curve. Also, the system can identify each DNA reaction model as either a major (or the first reaction model that is defined as being the closest reaction model to the WT CTRL reaction) or a secondary reaction model relating to the mutation.

FIG. 18B illustrates an example embodiment of a comparison of a wild-type control sample's negative derivative curve and a tested unknown sample's background-corrected negative derivative curve.

FIG. 18C illustrates example embodiments of temperature boundaries. This example uses the negative derivative curve from FIG. 16B as the WT control sample's negative derivative curve. This example also includes three mutations' boundaries as contained in the configuration information: one for a non-target HOM, one for a target WT, and one for a target HOM. An automatic genotyping system may provide a genotyping decision based on the overlap of the temperature difference points within any of the defined genotype temperature boundaries.

FIG. 19 illustrates an example embodiment of an automatic genotyping system. The system includes a genotyping device 1900 and an image-capturing device 1912. In this embodiment, the devices communicate by means of one or more networks 1999, which may include a wired network, a wireless network, a LAN, a WAN, a MAN, and a PAN. Also, in some embodiments the devices communicate by means of other wired or wireless channels.

The genotyping device 1900 includes one or more processors 1901, one or more I/O interfaces 1902, and storage 1903. Also, the hardware components of the genotyping device 1900 communicate by means of one or more buses or other electrical connections. Examples of buses include a universal serial bus (USB), an IEEE 1394 bus, a PCI bus, an Accelerated Graphics Port (AGP) bus, a Serial AT Attachment (SATA) bus, and a Small Computer System Interface (SCSI) bus.

The one or more processors 1901 include one or more central processing units (CPUs), which include microprocessors (e.g., a single core microprocessor, a multi-core microprocessor), one or more graphics processing units (GPUs), or other electronic circuitry. The one or more processors 1901 are configured to read and perform computer-executable instructions, such as instructions that are stored in the storage 1903 (e.g., ROM, RAM, a module). The I/O interfaces 1902 include communication interfaces to input and output devices, which may include a keyboard, a display device, a mouse, a printing device, a touch screen, a light pen, an optical-storage device, a scanner, a microphone, a camera, a drive, a controller (e.g., a joystick, a control pad), and a network interface controller.

The storage 1903 includes one or more computer-readable storage media. A computer-readable storage medium, in contrast to a mere transitory, propagating signal per se, includes a tangible article of manufacture, for example a magnetic disk (e.g., a floppy disk, a hard disk), an optical disc (e.g., a CD, a DVD, a Blu-ray), a magneto-optical disk, magnetic tape, and semiconductor memory (e.g., a non-volatile memory card, flash memory, a solid-state drive, SRAM, DRAM, EPROM, EEPROM). Also, as used herein, a transitory computer-readable medium refers to a mere transitory, propagating signal per se, and a non-transitory computer-readable medium refers to any computer-readable medium that is not merely a transitory, propagating signal per se. The storage 1903, which may include both ROM and RAM, can store computer-readable data or computer-executable instructions. The storage 1903 stores obtained configuration information 1903F, which can be received by means of one or more input devices or from another computing device by means of the network 1999.

The genotyping device 1900 also includes a preprocessing module 1903A, a Van't Hoff mixture-model-fitting module 1903B, a genotyping-decision module 1903C, an expectation-maximization (EM) module 1903D, and a communication module 1903E. A module includes logic, computer-readable data, or computer-executable instructions, and may be implemented in software (e.g., Assembly, C, C++, C#, Java, BASIC, Perl, Visual Basic), hardware (e.g., customized circuitry), or a combination of software and hardware. In some embodiments, the devices in the system include additional or fewer modules, the modules are combined into fewer modules, or the modules are divided into more modules. When the modules are implemented in software, the software can be stored in the storage 1903.

The preprocessing module 1903A includes instructions that, when executed, or circuits that, when activated, cause the genotyping device 1900 to perform preprocessing on HRM data based on the configuration information 1903F, thereby generating one or more preprocessed melting curves, or to calculate CQI noise (e.g., as performed in block B200 of FIG. 2 or blocks B1006, B1024, and B1044 of FIG. 10).

The mixture-model-fitting module 1903B includes instructions that, when executed, or circuits that, when activated, cause the genotyping device 1900 to fit one or more melting curves to a mixture model, thereby generating a background-corrected melting curve, or to calculate a CQI fit (e.g., as performed in block B210 of FIG. 2 or blocks B1008, B1026, and B1046 of FIG. 10).

The genotyping-decision module 1903C includes instructions that, when executed, or circuits that, when activated, cause the genotyping device 1900 to determine a genotype of an unknown sample's melting curve (e.g., a background corrected melting curve) based on the unknown sample's melting curve and on one or more of a WT control sample's melting curve and an ITC sample's melting curve, to generate a genotype probability, and to generate a CQI (e.g., as performed in block B220 of FIG. 2 or blocks B1014, B1032, B1054, and B1056 of FIG. 10).

The EM module 1903D includes instructions that, when executed, or circuits that, when activated, cause the genotyping device 1900 to perform an EM operation, for example as described in FIG. 15. Additionally, the EM module 1903D may be part of the mixture-model-fitting module 1903B.

The communication module 1903E includes instructions that, when executed, or circuits that, when activated, cause the genotyping device 1900 to communicate with one or more other devices, for example to obtain HRM data (e.g., melting curves) and to obtain configuration information. In some embodiments, the communication module 1903E implements a web-based function that allows users to upload data for their own assays and train the genotyping-decision module 1903C to determine the genotype class an unknown sample using HRM data that was generated by the assay.

The image-capturing device 1912 includes one or more processors 1913, one or more I/O interfaces 1914, and storage 1915. The image-capturing device also includes a communication module 1915A. The communication module 1915A includes instructions that, when executed, or circuits that, when activated, cause the image-capturing device 1912 to communicate with the genotyping device 1900, for example to send HRM data to the genotyping device 1900.

Additionally, the image-capturing device 1912 includes an image-capturing assembly 1916. The image-capturing assembly 1916 includes one or more image sensors that capture high-resolution fluorescence information from samples that are undergoing a melting process. The image-capturing assembly 1916 may also include one or more lenses and illumination devices.

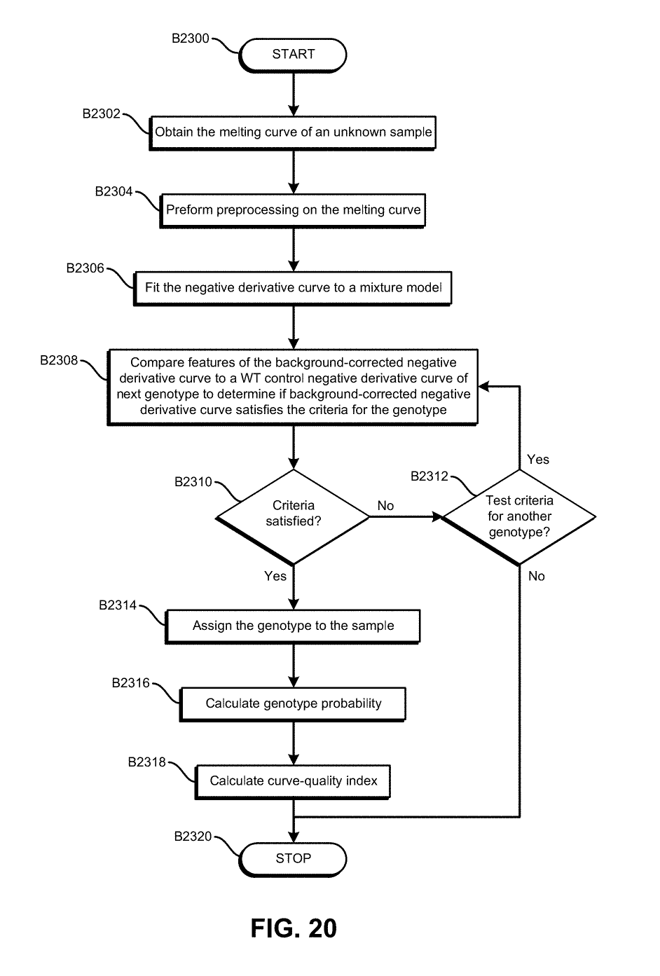

FIG. 20 illustrates an example embodiment of an operational flow for assigning a genotype to a sample. The operational flow may be performed by one or more specially-configured systems or devices (e.g., the automatic genotyping system in FIG. 1, the automatic genotyping system in FIG. 19). The flow starts in block B2000 and moves to block B2002, where the melting curve of an unknown sample is obtained. The melting curve may be the original −dF/dT curve in FIG. 16A, the original −dF/dT curve in FIG. 17A, and the original −dF/dT curve in FIG. 18A, or the melting curve may show raw fluorescence versus temperature instead of the negative derivative of fluorescence with respect to temperature. Next, in block B2004, preprocessing is performed on the melting curve. The preprocessing may remove noise from the melting curve. In some embodiments, the operations in block B2002 use raw fluorescence data, and the operations in block B2004 also calculate a negative derivative curve (−dF/dT) based on the raw fluorescence data.

The flow then moves to block B2006, where the negative derivative curve is fit to the mixture model, and then the background reaction curve is removed from the original negative derivative curve, thereby generating a background-corrected negative derivative curve (e.g., the reaction model in FIG. 16A, the reaction model in FIG. 17A, and the first and second reaction models in FIG. 18A).

The flow then proceeds to block B2008, where characteristics of the background-corrected negative derivative curve are compared to the WT control negative derivative curve to determine if the background-corrected negative derivative curve satisfies the criteria for the genotype.

The flow then moves to block B2010, where the one or more systems or devices determine if all criteria are satisfied. If not, then the flow moves to block B2012, where the systems or devices determine if the criteria for another genotype should be tested. If not, then the flow moves to block B2020, where the flow ends. If yes, then the flow returns to block B2008, where the criteria for another genotype are evaluated.

If in block B2010 the systems or devices determine that the criteria for the genotype are satisfied, then the flow moves to block B2014. In block B2014, the genotype is assigned to the sample. Next, in block B2016, the genotype probability is calculated. The flow then proceeds to block B2018, where the curve-quality index is calculated, and finally the flow ends in block B2020.

At least some of the above-described devices, systems, and methods can be implemented, at least in part, by providing one or more computer-readable media that contain computer-executable instructions for realizing the above-described operations to one or more genotyping devices that are configured to read and execute the computer-executable instructions. The systems or devices perform the operations of the above-described embodiments when executing the computer-executable instructions. Also, an operating system on the one or more systems or devices may implement at least some of the operations of the above-described embodiments.

Furthermore, some embodiments use one or more functional units to implement the above-described devices, systems, and methods. The functional units may be implemented in only hardware (e.g., customized circuitry) or in a combination of software and hardware (e.g., a microprocessor that executes software).

The scope of the claims is not limited to the above-described embodiments and includes various modifications and equivalent arrangements. Also, as used herein, the conjunction “or” generally refers to an inclusive “or,” though “or” may refer to an exclusive “or” if expressly indicated or if the context indicates that the “or” must be an exclusive “or.”

What is claimed is:

1. A system for genotyping a target nucleic acid in a test sample, the system comprising:
a microfluidic device having the test sample and a control sample, the control sample including wild type of the target nucleic acid;
one or more image-capturing devices configured to acquire images of the test and control samples to provide high-resolution melt data; and
one or more processors coupled to a computer-readable media and in communication with the one or more image-capturing devices, the one or more processors configured to cause the system to:
obtain high-resolution melt data from the test sample defining a melting curve for the target nucleic acid in the test sample;
obtain high-resolution melt data from the control sample defining a melting curve for the wild type of the target nucleic acid in the control sample;
calculate melting curve derivatives of the melting curves for the test sample and the control sample, respectively, wherein each melting curve derivative represents a negative derivative of a fluorescence emitted from a nucleic acid sample as a function of temperature affecting nucleic acid denaturation;
calculate parameters defining differences between features of the test sample and the control sample melting curve derivatives; and
assign a genotype to the test sample based on a comparison of the calculated parameters to predetermined thresholds and boundaries defining genotypes.

2. The system of claim 1, wherein the test sample and the control sample include an internal temperature control (ITC) component.

3. The system of claim 1, wherein the one or more processors are further configured to cause the system to
remove one or more background-reaction components from the test sample melting curve derivative and from the control sample melting curve derivative.

4. The system of claim 3, wherein the one or more background-reaction components are identified and removed from each of the test sample and the control sample melting curve derivatives by applying the Van't Hoff mixture model.

5. The system of claim 2, wherein the test sample is assigned a genotype only if the ITC reaction component is determined to be valid.

6. The system of claim 1, wherein the thresholds and boundaries are defined by a training set containing a sufficient number of samples revealing each specific genotype and variant associated with a specific assay.

7. The system of claim 1, wherein one-side portions of the test sample and the control sample melting curve derivatives are compared to determine differences if a mixture model for the test sample reveals only one reaction model, the one-side portion of each curve being defined as the portion to the left-side or right-side of a reaction peak of a melting curve derivative.

8. The system of claim 1, wherein relative positioning of reaction peaks in the test sample and in the control sample melting curve derivatives determines whether to perform a left-sided or right-sided comparison of the test sample and the control sample melting curve derivatives.

9. The system of claim 1, wherein the genotype is selected from the group consisting of: homozygous (HOM), heterozygous (HET), and wild type.

10. The system of claim 1, wherein calculating parameters defining differences between specific features of the test sample and the control sample melting curve derivatives includes determining a maximum fluorescence difference, ΔFₚ, between left-side portions of the test sample and the control sample melting curve derivatives.

11. The system of claim 10, wherein assigning the genotype to the test sample based on a comparison of the calculated parameters to predetermined thresholds and boundaries defining genotypes includes:
considering, for the test sample, a HET genotype if ΔFₚ≧ΔF0; and
considering WT or HOM as a potential genotype for the test sample if ΔFₚ<ΔF0, wherein ΔF0 is a predetermined threshold.

12. The system of claim 11, wherein if ΔFₚ≧ΔF0, and the difference between a temperature where ΔFₚ occurs and a temperature of a major reaction peak of the test sample melting curve derivative, ΔTₚ , is within the defined HET boundaries, then the test sample is assigned to HET, where the major reaction peak is identified as the closest peak to a control sample peak of the control sample melting curve derivative.

13. The system of claim 1, wherein a noise signal index is calculated for each melting curve derivative prior to comparing the melting curve derivatives to the predetermined thresholds.

14. The system of claim 1, wherein the one or more processors are further configured to cause the system to generate a genotype probability based upon parameters defining differences between features of the test sample melting curve derivative and the control sample melting curve derivative and define the predetermined thresholds.

15. The system of claim 1, wherein the microfluidic device has a non-template control (NTC) sample.

16. A method for genotyping a target nucleic acid in a test sample, the method comprising:
providing a microfluidic device having the test sample and a control sample, the control sample including a wild type of the target nucleic acid;
providing one or more image-capturing devices configured to acquire images of the test and the control samples to provide high-resolution melt data; and
providing one or more processors coupled to a computer-readable media and in communication with the one or more image-capturing devices, the computer-readable media comprising instructions for:
obtaining high-resolution melt data from the test sample defining a melting curve for the target nucleic acid in the test sample;
obtaining high-resolution melt data from the control sample defining a melting curve for the wild type nucleic acid in the control sample;
calculating melting curve derivatives of the melting curves for the test sample and the control sample, respectively, wherein each melting curve derivative represents a negative derivative of a fluorescence emitted from a nucleic acid sample as a function of temperature causing nucleic acid denaturation;
calculating parameters defining differences between features of the test sample and the control sample melting curve derivatives; and
assigning a genotype to the test sample based on a comparison of the calculated parameters to predetermined thresholds and boundaries defining genotypes.

17. The method of claim 16, wherein the test sample and the control sample include an internal temperature control (ITC) component.

18. The system of claim 16, wherein the computer-readable media comprises further instructions for removing one or more background-reaction components from the test sample melting curve derivative and from the control sample melting curve derivative, thereby generating background-corrected melting curve derivatives for calculating parameters defining differences between features of the test sample and the control sample.

19. The method of claim 18, wherein the one or more background-reaction components are identified and removed from each of the test sample and the control sample melting curve derivatives using a Van't Hoff mixture model.

20. The method of claim 17, wherein the test sample is assigned the genotype only if the ITC reaction component is determined to be valid.

21. The method of claim 16, wherein the predetermined thresholds and class boundaries are defined by a training set containing a sufficient number of samples revealing each specific genotype and variant associated with a specific assay.

22. The method of claim 16, wherein one-side portions of the test sample and the control sample melting curve derivatives are compared if a mixture model for the test sample reveals only one reaction model, the one-side portion of each curve being defined as the portion to the left or right of a reaction peak of a melting curve derivative.

23. The method of claim 16, wherein relative positioning of reaction peaks determines whether to perform a left-sided or right-sided comparison of the test sample and the control sample melting curve derivatives.

24. The method of claim 16, wherein the genotype is selected from the group consisting of: homozygous (HOM), heterozygous (HET), and wild type.

25. The method of claim 16, wherein calculating parameters defining differences between specific features of the test sample and the control sample melting curve derivatives includes determining a maximum fluorescence difference, ΔFₚ, between left-side portions of the test sample and the control sample melting curve derivatives.

26. The method of claim 25, wherein assigning the genotype to the test sample based on a comparison of the calculated parameters to the predetermined thresholds and boundaries defining genotypes includes:
Considering, for the test sample, a HET genotype if ΔFₚ≧ΔF0; and
considering WT or HOM as a potential genotype for the test sample if ΔFₚ<ΔF0, wherein ΔF0 is a predetermined threshold.

27. The method of claim 26, wherein if ΔFₚ≧ΔF0, and the difference between a temperature where ΔFₚ occurs and a temperature of a major reaction peak of the test sample melting curve derivative, ΔTₚ , is within defined HET boundaries, then the test sample is assigned to HET, where the major reaction peak is identified as the closest peak to a control sample peak of the control sample melting curve derivative.

28. The method of claim 16, wherein a noise signal index is calculated for each melting curve derivative prior to comparing the melting curve derivatives to the predetermined thresholds.

29. The method of claim 16, wherein the one or more processors are further configured to cause the system to generate a genotype probability based upon parameters defining differences between features of the test sample and control sample melting curve derivatives and the predetermined thresholds.

30. The method of claim 16, wherein the microfluidic device has a non-template control (NTC) sample.
